(19) United States
(12) Patent Application Publication
(10) Pub. No.: US 2024/0398945 A1
(43) Pub. Date: Dec. 5, 2024

(54) CD19-SPECIFIC CHIMERIC ANTIGEN RECEPTOR T-CELL THERAPY

(71) Applicants: FUNDACIÓ DE RECERCA CLINIC BARCELONA-INSTITUT D'INVESTIGACIONS BIOMÈDIQUES AUGUST PI I SUNYER, Barcelona (ES); HOSPITAL CLÍNIC DE BARCELONA, Barcelona (ES); UNIVERSITAT DE BARCELONA, Barcelona (ES)

(72) Inventors: Manel JUAN OTERO, Barcelona (ES); Álvaro URBANO ISPIZUA, Barcelona (ES); Mariona PASCAL CAPDEVILA, Barcelona (ES); Jordi YAGÜE RIBES, Barcelona (ES); Julio DELGADO GONZÁLEZ, Barcelona (ES); Jordi ESTEVE REYNER, Barcelona (ES); María CASTELLÀ CASTELLÀ, Barcelona (ES); Ana BORONAT BARADO, Barcelona (ES)

(21) Appl. No.: 18/558,895

(22) PCT Filed: May 6, 2022
(86) PCT No.: PCT/EP2022/062374

§ 371 Date: Nov. 3, 2023

(30) Foreign Application Priority Data
May 6, 2021 (WO) PCT/ES2021/070316

(51) Int. Cl. A61K 39/00; A61P 35/00; A61P 35/02

(52) U.S. Cl. CPC A61K 39/4611; A61K 39/4631; A61K 39/464412; A61P 35/00; A61P 35/02; A61K 2239/38; A61K 2239/48

(57) ABSTRACT

The present invention refers to a CD19-specific chimeric antigen receptor (CAR) T-cell therapy and to its use for treating CD19+ malignancies.

### FIELD OF THE INVENTION

The present invention refers to the medical field. Particularly, the present invention refers to a CD19-specific chimeric antigen receptor (CAR) T-cell therapy based on a new scFv and to its use for treating CD19+ malignancies.

### STATE OF THE ART

Genetically modifying autologous T cells to express CARs, thus redirecting them to eliminate tumor cells, is a revolutionary therapeutic modality for cancer treatment and, in particular, for CD19+ B-cell malignancies.

CARs are composed of an extracellular region responsible for binding to a particular antigen and an intracellular region that promotes T cell cytotoxic activity and proliferation. CAR binding to the selected antigen is usually mediated by a single chain variable fragment (scFv) of a monoclonal antibody. The scFv-derived region results in a medium-high affinity and MHC-independent interaction of the CAR with its ligand. As a second-generation CAR, this scFv is combined with an intracellular co-stimulatory domain (usually CD28 or 4-1BB) and a pro-activator cytotoxic domain (CD3z).

After initial disappointing results with first-generation CARs, the most recent clinical trials with second generation anti-CD19 CAR T cells have shown remarkable results in patients with chronic lymphocytic leukemia, non-Hodgkin's lymphoma and acute lymphoid leukemia (ALL). Several academic groups, including the University of Pennsylvania, Memorial Sloan Kettering Cancer Center, National Cancer Institute and the Fred Hutchinson Cancer Research Center, pioneered these seminal studies using slightly different CAR constructs that are currently under evaluation in a number of international multi-center clinical trials. Two of these CAR products (tisagenlecleucel and axicabtagene ciloleucel) based on the scFv derived of the monoclonal antibody named FMC63 clone were recently approved by the US Food and Drug Administration and European Medicine Agency for clinical use. Regarding efficacy, response rates range from 50 to 85%, depending on the type of B-cell malignancy and CAR construct, with quite remarkable disease-free and overall survival. In terms of safety, patients who respond to therapy usually develop persistent B-cell aplasia and transitory cytokine release syndrome which could be severe in a small proportion of patients.

Despite these striking results, this therapeutic approach is only available in a handful of centers and it is uncertain when and at what cost this could be available elsewhere.

Thus, the present invention is focused on developing a new CD19-specific chimeric antigen receptor T-cell therapy based on a new scFv different from monoclonal antibody named FMC63 clone for the treatment of CD19+ malignancies.

### DESCRIPTION OF THE INVENTION

### Brief Description of the Invention

As explained above, the present invention refers to a CD19-specific chimeric antigen receptor (CAR) T-cell therapy and its use for treating CD19+ malignancies.

According to the results provided in the present invention (see Example 1), CART-cells of the invention are highly cytotoxic against CD19+ cells in vitro, inducing secretion of pro-inflammatory cytokines and CART-cell proliferation. In vivo, the CART cells of the invention are able to fully control disease progression in an NSG xenograph B-ALL mouse model. Based on the preclinical data, it can be concluded that the CART-cells of the invention are clearly functional against CD19+ cells.

On the other hand, the present invention shows (see Example 2) the production of 28 CAR T-cell products in the context of a phase I clinical trial for CD19+ B-cell malignancies. The system includes CD4-CD8 cell selection, lentiviral transduction and T-cell expansion using IL-7/IL-15. 27 out of 28 CAR T-cell products manufactured met the full list of specifications and were considered valid products. Ex vivo cell expansion lasted an average of 8.5 days and had a mean transduction rate of 30.6%=13.44. All products obtained presented cytotoxic activity against CD19+ cells and were proficient in the secretion of pro-inflammatory cytokines. Expansion kinetics was slower in patient's cells compared to healthy donor's cells. However, product potency was comparable. CAR T-cell subset phenotype was highly variable among patients and largely determined by the initial product. T_{CM} and T_{EM} were the predominant T-cell phenotypes obtained. 38.7% of CAR T-cells obtained presented a T_{N} or T_{CM} phenotype, in average, which are the subsets capable of establishing a long-lasting T-cell memory in patients. An in-depth analysis to identify individual factors contributing to the optimal T-cell phenotype revealed that ex vivo cell expansion leads to reduced numbers of T_{N}, T_{SCM} and T_{EFF} cells, while T_{CM} cells increase, both due to cell expansion and CAR− expression. Overall, these results show a feasible system for producing clinical-grade CAR T-cells for heavily pre-treated patients, and that the obtained products meet the current quality standards of the field. Reduced ex vivo expansion may yield CAR T-cell products with increased persistence in vivo.

Finally, it is of utmost importance to consider that the administration of the CAR T-cells of the invention has been evaluated and results confirming that the therapy is safe and effective have been obtained (see Example 3).

So, the first embodiment of the present invention refers to an antibody, F(ab)2, Fab, scFab or scFv (hereinafter antibody, F(ab′)2, Fab, scFab or scFv of the invention) comprising a light chain variable region (VL) and a heavy chain variable region (VH), wherein said VH comprises HCDR1, HCDR2 and HCDR3 polypeptides and VL comprises LCDR1, LCDR2 and LCDR3 polypeptides, and wherein HCDR1 consists of the sequence SEQ ID NO: 1, HCDR2 consists of the sequence SEQ ID NO: 2, HCDR3 consists of the sequence SEQ ID NO: 3, LCDR1 consists of the sequence SEQ ID NO: 4, LCDR2 consists of the sequence SEQ ID NO: 5 and LCDR3 consists of the sequence SEQ ID NO: 6.

Particularly, the complementarity-determining region (CDR) sequences are as follows:

HCDR1 (SEQ ID NO: 1):
FAFSSYWMNWV
HCDR2 (SEQ ID NO: 2):
GQIYPGDGDT
HCDR3 (SEQ ID NO: 3):
RKRITAVIT
LCDR1 (SEQ ID NO: 4):
RASESVDNFGNSFMH
LCDR2 (SEQ ID NO: 5):
IYIASNLES
LCDR3 (SEQ ID NO: 6):
HQNNEDPLTF

In a preferred embodiment, the antibody, F(ab′)2, Fab, scFab or scFv of the invention comprises a light chain variable region (VL domain) and a heavy chain variable region (VH domain), wherein the VL domain consists of SEQ ID NO: 7 and the VH domain consists of SEQ ID NO: 8.

Particularly, the VL and VH sequences are as follows:

VL
(SEQ ID NO: 7)
TGNIVLTQSPASLAVSLGQRATISCRASESVDNFGNSFMHWYQQKSGQP
PRLLIYIASNLESGVPARFSGSGSRTDFTLTIDPVEADDAATYYCHQNN
EDPLTFGAGTKLELK
Note: CDRs are underlined.
VH
(SEQ ID NO: 8)
HSQIQLQQSGAELVRPGSSVKISCKASGFAFSSYWMNWVKQRPGQGLEW
IGQIYPGDGDTKYNVKFRGKATLTADESSSTAYIQLTSLTSEDSGVYFC
ARKRITAVITTVFDVWGAGTTVTVSS
Note: CDRs are underlined.

The second embodiment of the present invention refers to a CAR (hereinafter CAR of the invention) comprising a scFv which in turn comprises a VL domain, a VH domain and a spacer, wherein the VL domain consists of SEQ ID NO: 7 and the VH domain consists of SEQ ID NO: 8.

In a preferred embodiment, the CAR of the invention further comprises a transmembrane domain, a costimulatory signalling domain and/or an intracellular signalling domain.

In a preferred embodiment, the hinge and transmembrane domain consists of CD8a of SEQ ID NO: 9, the costimulatory signalling domain consists of 4-1BB of SEQ ID NO: 10 and the intracellular signalling domain consists of CD38 of SEQ ID NO: 11.

Particularly, the CD8a, 4-1BB and CD38 sequences are as follows:

CD8a
(SEQ ID NO: 9)
TTTPAPRPPTPAPTIASQPLSLRPEACRPAAGGAVHTRGLDFACDIYIW
APLAGTCGVLLLSLVITLYC
4-1BB
(SEQ ID NO: 10)
KRGRKKLLYIFKQPFMRPVQTTQEEDGCSCRFPEEEEGGCEL
CD38
(SEQ ID NO: 11)
RVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTAT
KDTYDALHMQALPPR

In a preferred embodiment, the CAR of the invention comprises SEQ ID NO: 12.

Particularly, the sequence of the CAR of the invention is as follows:

CAR
(SEQ ID NO: 12)
[VL + spacer + VH + CD8a + 4-1BB + CD38]
TGNIVLTQSPASLAVSLGQRATISCRASESVDNFGNSFMHWYQQKSGQP
PRLLIYIASNLESGVPARFSGSGSRTDFTLTIDPVEADDAATYYCHQNN
EDPLTFGAGTKLELKGGGGSGGGGSGGGGSGGGGSHSQIQLQQSGAELV
RPGSSVKISCKASGFAFSSYWMNWVKQRPGQGLEWIGQIYPGDGDTKYN
VKFRGKATLTADESSSTAYIQLTSLTSEDSGVYFCARKRITAVITTVFD
VWGAGTTVTVSSTTTPAPRPPTPAPTIASQPLSLRPEACRPAAGGAVHT
RGLDFACDIYIWAPLAGTCGVLLLSLVITLYCKRGRKKLLYIFKQPFMR
PVQTTQEEDGCSCRFPEEEEGGCELRVKFSRSADAPAYQQGQNQLYNEL
NLGRREEYDVLDKRRGRDPEMGGKPQRRKNPQEGLYNELQKDKMAEAYS
EIGMKGERRRGKGHDGLYQGLSTATKDTYDALHMQALPPR
Note: CDRs are underlined.

The third embodiment of the present invention refers to a nucleic acid encoding the CAR of the invention, preferably a nucleic acid comprising SEQ ID NO: 13.

Particularly, the sequence of the nucleic acid encoding the CAR of the invention is as follows:

Lentivirus vector
(SEQ ID NO: 13)
[EF1A promoter + CD8 peptide leader + VL + spacer + VH + CD8a + 4-1BB + CD38]
CGCCTTTTTCCGGAGGGTGGGGGAGAACCGTATATAAGTGCAGTAGTCCGTGAA
CGTTCTTTTTCGCAACGGGTTTGCCGCCAGAACACAGGTAAGTGCCGTGTGTGGT
TCCCGCGGGCCTGGCCTCTTTACGGGTTATGGCCCTTGCGTGCCTTGAATTACTTC
CACCTGGCTGCAGTACGTGATTCTTGATCCCGAGCTTCGGGTTGGAAGTGGGTGG
GAGAGTTCGAGGCCTTGCGCTTAAGGAGCCCCTTCGCCTCGTGCTTGAGTTGAGG
CCTGGCCTGGGCGCTGGGGCCGCCGCGTGCGAATCTGGTGGCACCTTCGCGCCTG
TCTCGCTGCTTTCGATAAGTCTCTAGCCATTTAAAATTTTTGATGACCTGCTGCGA
CGCTTTTTTTCTGGCAAGATAGTCTTGTAAATGCGGGCCAAGATCTGCACACTGG
TATTTCGGTTTTTGGGGCCGCGGGCGGCGACGGGGCCCGTGCGTCCCAGCGCACA
TGTTCGGCGAGGCGGGGCCTGCGAGCGCGGCCACCGAGAATCGGACGGGGGTAG
TCTCAAGCTGGCCGGCCTGCTCTGGTGCCTGGCCTCGCGCCGCCGTGTATCGCCC
CGCCCTGGGCGGCAAGGCTGGCCCGGTCGGCACCAGTTGCGTGAGCGGAAAGAT
GGCCGCTTCCCGGCCCTGCTGCAGGGAGCTCAAAATGGAGGACGCGGCGCTCGG
GAGAGCGGGCGGGTGAGTCACCCACACAAAGGAAAAGGGCCTTTCCGTCCTCAG
CCGTCGCTTCATGTGACTCCACTGAGTACCGGGCGCCGTCCAGGCACCTCGATTA
GTTCTCGTGCTTTTGGAGTACGTCGTCTTTAGGTTGGGGGGAGGGGTTTTATGCG
ATGGAGTTTCCCCACACTGAGTGGGTGGAGACTGAAGTTAGGCCAGCTTGGCAC
TTGATGTAATTCTCCTTGGAATTTGCCCTTTTTGAGTTTGGATCTTGGTTCATTCTC
AAGCCTCAGACAGTGGTTCAAAGTTTTTTTCTTCCATTTCAGGTGTCATGA
GCCTCATCCCGAATG GCT CTG CCT GTC ACT GGA CTG CTG CTG TCA CTG GGG
CTG CTG CTG CAC GCC GCA AGA CCA
ACC GGT AAC ATC GTG CTG ACT CAG AGC CCA GCA TCC CTG GCC GTC TCA
CTG GGA CAG CGG GCT ACC ATC AGC TGC AGA GCA AGC GAG TCC GTG GAC
AAC TTC GGA AAT TCC TTT ATG CAC TGG TAC CAG CAG AAG TCT GGC CAG
CCC CCT CGA CTG CTG ATC TAT ATT GCC TCT AAC CTG GAG AGT GGC GTG
CCA GCT AGG TTC TCT GGC AGT GGG TCA CGC ACA GAC TTT ACC CTG ACA
ATT GAT CCC GTC GAA GCC GAC GAT GCC GCT ACC TAC TAT TGC CAC CAG
AAC AAC GAG GAC CCA CTG ACT TTC GGA GCA GGG ACA AAA CTG GAA CTG
AAG
GGTGGAGGCGGGAGC GGAGGAGGCGGGTCG GGAGGAGGAGGATCG
GGTGGCGGAG GCTCA
CAT TCT CAG ATT CAG CTG CAG CAG TCAGGG GCA GAG CTG GTG CGG CCA
GGG TCC TCA GTC AAG ATT AGT TGT AAG GCT TCA GGC TTT GCT TTC AGC
TCC TAC TGG ATG AAC TGG GTG AAA CAG CGA CCA GGA CAG GGA CTG GAG
TGG ATC GGA CAG ATC TAC CCC GGA GAC GGC GAT ACA AAG TAT AAT GTC
AAA TTT CGG GGC AAG GCC ACC CTG ACA GCT GAC GAG TCT AGT TCA ACC
GCC TAC ATC CAG CTG ACT TCA CTG ACC AGC GAA GAT TCC GGC GTG TAT
TTC TGC GCT CGA AAG CGG ATC ACT GCA GTC ATT ACC ACA GTG TTT GAC
GTG TGG GGG GCA GGA ACT ACC GTG ACC GTC AGC TCC
ACA ACT ACG CCG GCG CCG AGA CCA CCT ACA CCT GCA CCA ACT ATT GCC
TCT CAG CCA CTG AGT CTG CGC CCC GAG GCA TGT CGA CCT GCC GCT GGC
GGG GCT GTG CAC ACC AGG GGC CTA GAC TTC GCC TGC GAT ATC TATATT
TGG GCT CCA CTG GCA GGA ACC TGT GGC GTG CTG CTG CTG TCT CTG GTC
ATC ACA CTG TAC TGC
AAA AGA GGC AGG AAG AAA CTG CTG TAT ATT TTC AAG CAG CCC TTT ATG
AGA CCT GTG CAG ACA ACT CAG GAG GAA GAC GGG TGC AGC TGT AGG TTC
CCT GAG GAA GAG GAA GGA GGC TGT GAG CTG
CGC GTG AAA TTT TCT CGG AGT GCA GAT GCC CCA GCT TAC CAG CAG GGC
CAG AAC CAG CTG TAT AAC GAG CTG AAT CTG GGG CGG AGA GAG GAA TAC
GAC GTG CTG GAT AAG AGG CGC GGG CGA GAT CCA GAA ATG GGA GGA
AAA CCC CAG CGA CGG AAG AAC CCT CAG GAG GGA CTG TAC AAT GAA CTG
CAG AAG GAC AAA ATG GCA GAG GCC TAT TCC GAA ATC GGG ATG AAA GGA
GAA AGA AGG CGC GGC AAG GGG CAT GAT GGC CTG TAT CAG GGA CTG TCA
ACC GCA ACA AAA GAT ACT TAT GAT GCT CTG CAC ATG CAG GCT CTG CCC
CCG CGG TAA

The fourth embodiment of the present invention refers to a cell comprising the CAR of the invention or the nucleic acid encoding the CAR of the invention (hereinafter CAR cells of the invention).

In a preferred embodiment, the cell is a T-cell (hereinafter CART cells of the invention) or a NK-cell.

The fifth embodiment of the present invention refers to a pharmaceutical composition (hereinafter pharmaceutical composition of the invention) comprising a plurality of cells of the invention and, optionally, a pharmaceutically acceptable carrier or excipient.

The sixth embodiment of the present invention refers to the cells or the pharmaceutical composition of the invention, for use as a medicament, preferably in the treatment of CD19+ malignancies, more preferably in the treatment of acute lymphoblastic leukaemia, non-Hodgkin's lymphoma or chronic lymphocytic leukaemia or any CD19+ disorder.

Alternatively, this sixth embodiment refers to a method for treating CD19+ malignancies, more preferably acute lymphoblastic leukaemia, non-Hodgkin's lymphoma or chronic lymphocytic leukaemia or any CD19+ disorder, which comprises the administration of a therapeutically effective dose of the cells or the pharmaceutical composition of the invention.

In a preferred embodiment, a fractionated administration of the cell composition of the invention, particularly a progressive dose fractionation, was carried out. Progressive dose fractionation means that patients received at least two, preferably at least three fractions of the composition of the invention, wherein the percentage of cells administered is progressively increased in each consecutive fraction. In a preferred embodiment, the progressive dose fractionation comprises a first fraction (around 10%) on day 0, followed by the second (around 30%) and third (around 60%) fraction.

Thus, the first patients received a single intra-venous infusion of the composition of the invention, but, as a proof of concept of the progressive dose fractionation, the following patients received the first fraction (around 10%) on day 0, followed by the second (around 30%) and third (around 60%) fraction. The second fraction was preferably administered 24-48 hours after the first fraction, and the third fraction was preferably administered 24-48 hours after the second fraction, only if the patient had no signs or symptoms of CRS.

The reason for this progressive fractionated administration proposal was avoiding toxicity, since three cases of fatal toxicity were detected.

In a preferred embodiment, the total dose of the cell composition of the invention depend on the specific patient to be treated (as explained in the Examples). Typical doses are 0.1-5×10⁶ cells/Kg, preferably 0.5-5×10⁶ cells/Kg, more preferably 1-5×10⁶ cells/Kg. Preferably, the total dose is fractionated in three fractions as explained above.

For the purpose of the present invention the following terms are defined:
The term “comprising” means including, but not limited to, whatever follows the word “comprising”. Thus, the use of the term “comprising” indicates that the listed elements are required or mandatory, but that other elements are optional and may or may not be present.
By “consisting of” it is meant including, and limited to, whatever follows the expression “consisting of”. Thus, the expression “consisting of” indicates that the listed elements are required or mandatory, and that no other elements may be present.
“Pharmaceutically acceptable excipient or carrier” refers to a compound that may optionally be included in the compositions of the invention and that causes no significant adverse toxicological effects to the patient.
By “therapeutically effective dose” of a composition of the invention is intended an amount that, when administered as described herein, brings about a positive therapeutic response in a subject suffering from a CD19+ malignancy. The exact amount required will vary from subject to subject, depending on the age, the general condition of the subject, the severity of the condition being treated, the mode of administration, etc. An appropriate “effective” amount in any individual case may be determined by one of ordinary skill in the art using routine experimentation, based upon the information provided herein.
“Fab” are antibody fragments with a size of around 50 KDa are the antigen-binding domains of an antibody molecule, containing one constant and one variable domain of each of the heavy and the light chains. The fragments which contain disulfide bridge thiols are called “Fab′ fragments”, whereas those lacking the thiol functional group are termed “Fab fragments”. To produce “Fab” fragments, two different methods can be employed. The primary method is via enzymatic/chemical cleavage of the whole antibody, in which the whole antibody is cleaved by enzyme (such as papain, pepsin, and ficin) to form “F(ab′)2” fragments, followed by the reduction of those fragments to yield “Fab” fragments. An alternative method is through the recombinant synthesis of “F(ab′)2” antibody fragments, followed by chemical reduction of these fragments to yield Fab units. A “F(ab′)2” fragment, which retains a small part of the Fc hinge region, has two antigen binding regions that may increase the affinity to antigen. Reduction of “F(ab′)2” fragments produces two monovalent Fab′ fragments, which have a free sulfhydryl group that is useful for conjugation with other molecules. Although the utilization of enzymatic/chemical cleavage methods to generate “Fab” fragments is convenient and efficient, it requires a large quantity of monoclonal antibody as starting material. A single chain “Fab” fragment (scFab) can lead to improved function and production of “Fab” fragments. According to some studies, “scFab” fragments show superior antigen-binding ability compared to “Fab” and compensate for some of the disadvantages of the soluble Fab production in E. coli.
The term “single-chain variable fragment” or “scFv” refers to a fusion protein comprising the variable domains of the heavy chain (VH) and light chain (VL) of an antibody linked to one another with a peptide linker. The term also includes a disulfide stabilized Fv (dsFv). Methods of stabilizing scFvs with disulfide bonds are disclosed in Reiter et al., 1996. Nat Biotechnol. 14 (10): 1239-45.
The expression “progressive dose fractionation” means that patients received at least two, preferably at least three fractions of the composition of the invention, wherein the percentage of cells administered is progressively increased in each consecutive fraction. In a preferred embodiment, the progressive dose fractionation comprises a first fraction (around 10%) on day 0, followed by the second (around 30%) and third (around 60%) fraction.

### DESCRIPTION OF THE FIGURES

FIG. 1. In vitro anti-tumor activity of the CAR of the invention. (A) Cytotoxicity assay of CART19 cells vs NALM6 cells. Percent target surviving cells, relative to untreated, is shown (Mean of 3 experiments±SEM). Panels on the right show representative flow cytometry plots at E:T ratio=1:8 (B) CAR19 T cell proliferation in vitro measured by CFSE assay. Panels on the left show representative flow cytometry images. Panel on the right shows quantification of Proliferation Index (PI). Mean of 3 experiments±SEM is shown. (C) Cytokine production (IFNγ, TNFα and IL-10) of CART19 cells in co-culture with NALM6 cells, measured by ELISA. Mean of 3 experiments±SEM is shown. (*) indicates statistical significance, p<0.05. n.s. indicates not statistically significant.

FIG. 2. In vivo anti-tumor activity of the CAR of the invention. (A) Upper panel shows a timeline of experimental design. Lower panels show bio-luminiscent images showing disease progression at different days. Animals indicated by (#) were sacrificed at day 16 due to advanced disease progression. The rest of the animals were sacrificed at day 17. (B) Detection of tumor (CD19+) cells in the bone marrow of mice shown in (a) (Mean±SD). (C) Detection of tumor (CD19+) cells in blood.

FIG. 3. Comparison of antitumor activity of the CART cells of the invention with FMC63-based CART cells. Upper panel shows a timeline of experimental design (*Im, bioluminescent image; *BI, blood sample). Lower panels show bioluminescence images showing disease progression at different days.

FIG. 4. Expansion of the CAR cells of the invention in CliniMACS Prodigy. (A) Expansion kinetics of CAR cells of the invention (Total cell number). Gray points indicate individual products. Black triangles indicate Mean±SD and adjusting curve. (B) Expansion kinetics of CAR19+ cells (red) and total cell number (black). Mean±SD is represented. (C) Expansion kinetics of CAR cells of the invention (Total cell number) comparing healthy controls and different types of disease. Mean±SEM is represented. (D) Percentage of CD3 and CAR19 positive cells as determined by flow cytometry. Mean±SD is also indicated. Panels on the right show flow cytometry representative image corresponding to CAR19 and CD3 staining in CAR cells of the invention (final products) and Control T cells (Untransduced).

FIG. 5. Cell potency of the CAR cells of the invention. (A) Cytotoxicity assay after 4 h of CAR cells of the invention co-culture with NALM6 cells, at the indicated ratios. Mean±SD of all 27 CAR T cell products is indicated. (#) Dashed line indicates minimum of CAR cells of the invention cytotoxicity level for a product to be considered valid. (B) IFNγ, TNFα and GranzymeB levels measured in the supernatants of the cytotoxicity assays. E:T ratio 0 indicates no target cells. (*) indicates statistical significance, p<0.05. (C) Comparison of CAR cells of the invention cytotoxic potential after 4 h of co-culture with NALM6 cells, at the indicated ratios. Mean±SD is shown. “n.s.” indicates not statistically significant (Non-parametric test). (D) Comparison of IFNγ, TNFα and GranzymeB levels measured in the supernatants of the cytotoxicity assay at E:T ratio 1:1. “HD” indicates Healthy donors. “n.s.” indicates not statistically significant (Parametric test applied to IFNγ and TNFα and non-parametric test applied to GranzymeB).

FIG. 6. Subset characterization of the CAR cells of the invention. (A) CD4/CD8 ratio of apheresis products, after CD4-CD8 cell selection and of the final product. (B) CD4/CD8 ratio variation during cell expansion. Left panel corresponds to products with an initial ratio <1. Right panel corresponds to products with an initial ratio >1. (C) CAR19 transduction efficiency in CD4 and CD8 cells. Mean±SD is shown. (D) Percentage of T-cell subpopulations within initial (CD4-CD8 cell selection) and final products (CAR− and CAR+ cells). (E) Differences in MFI for CD45RA and CCR7 in initial and final products. Lower panel shows paired analysis for CCR7 MFI. (*) indicates statistical significance, p<0.05. n.s. indicates not statistically significant.

FIG. 7. Clinical outcome of patients with Acute Lymphoblastic Leukaemia. (A-D) Progression-free survival (A), overall survival (B), in vivo survival of CAR cells of the invention, as measured by persistence of B cell aplasia (C), and procedure-related mortality (D) of patients with acute lymphoblastic leukaemia belonging to the modified full analysis set (n=38) according to type of administration (cohorts 1 and 2 versus cohort 3).

### DETAILED DESCRIPTION OF THE INVENTION

The present invention is illustrated by means of the Examples set below without the intention of limiting its scope of protection.

### EXAMPLES

### Example 1. Development of the Anti-CD19 CAR of the Invention

### Example 1.1. Material and Methods

### Example 1.1.1. Donors, Cell Lines and Anti-CD19 Monoclonal Antibody

All protocols were approved by the corresponding Institutional Review Board. Healthy donor blood buffy-coats were obtained from the local reference blood bank (Banc de Sang i Teixits, Barcelona). NALM6, HL60, K562 and 300. 19 cell lines were purchased from the American Type Culture Collection (ATCC). These four cell lines were cultured in RPMI media+10% FBS+antibiotics. HEK293T cell line was also purchased from the ATCC (#CRL-11268) and cultured in DMEM+10% FBS+antibiotics. All cell lines were grown at 37° C. and 5% CO₂. 300.19-hCD19 stable transfected cells were produced using pUNO1-CD19 cDNA (InvivoGen, San Diego, CA).

The murine anti-CD19 monoclonal antibody of the invention was generated at Department of Immunology (Hospital Clínic de Barcelona) and its anti-CD19 specificity was confirmed.

### Example 1.1.2. CAR19 Cloning and Lentivirus Production

The sequence corresponding to the VL and VH regions of the antibody of the invention was extracted from hybridoma cells using Mouse Ig-Primer Set (Novagen, Cat. N. 69831-3). The complete CAR19 sequence (including signal peptide, antibody scFv, CD8 hinge and transmembrane regions, 4-1BB and CD3z) was synthesized by GeneScript and cloned into the 3^{rd} generation lentiviral vector pCCL (kindly provided by Dr. Luigi Naldini; San Raffaele Hospital, Milan), under the control of EF1α promoter.

To produce lentiviral particles for pre-clinical studies, HEK293T cells were transfected with our transfer vector (pCCL-EF1α-CAR19) together with packaging plasmids pMDLg/pRRE (Addgene, #12251), pRSV-Rev (Addgene, #12253) and envelope plasmid pMD2.G (Addgene, #12259), using linear polyethylenimine MW 25000 (PEI) (Polysciences, Cat. N. 23966-1). Briefly, 6×10⁶ HEK293T cells were plated 24 h before transfection in 10 cm-dishes. At the time of transfection 14 μg of total DNA (6.9 μg transfer vector, 3.4 μg pMDLg/pRRE, 1.7 μg pRSV-Rev and 2 μg pMD2.G) was diluted in serum-free DMEM. 35 μg of PEI was added to the mix and incubated 20 min at room temperature. After incubation, DNA-PEI complexes were added on to the cells cultured in 7 ml complete DMEM media. Media was replaced 4 h later. Viral supernatants were collected 48 h later and clarified by centrifugation and filtration using a 0.45 μm filter. Viral supernatants were concentrated using ultracentrifugation at 26,000 rpm for 2 h 30 min. Virus-containing pellets were resuspended in complete XVivo15 media and stored at −80° C. until use.

### Example 1.1.2. Lentivirus Titration

The number of transducing units (TU/ml) was determined by limiting dilution method. Briefly, HEK293T cells were seeded 24 h before transduction. Then, 1:10 dilutions of the viral supernatant were prepared and added on top of the cells in complete DMEM media+5 μg/ml Polybrene. Cells were trypsinyzed 48 h later and labeled with APC-conjugated AffiniPureF(ab′)₂ Fragment Goat anti-mouse IgG (Jackson Immunoresearch. Cat. N. 115-136-072) before being analyzed by flow cytometry. Dilution corresponding to 2-20% of positive cells was used to calculate viral titer.

### Example 1.1.3. T-Cell Transduction and Culture Conditions

Healthy donor PBMCs were obtained from buffy-coats by density-gradient centrifugation (Ficoll) after donation consented following instruction of Ethics Committee. While monocytes were eliminated by conventional plate adhesion, the remaining cells were cultured in X-VIVO 15 Cell Medium (Cultek, #BE02-060Q), 5% AB human serum (Sigma, Cat. N. H4522), penicillin-streptomycin (100 μg/ml) and IL-2 (50 IU/ml; Miltenyi Biotec). Cells were then activated and expanded for 24 h using beads conjugated with CD3 and CD28 MAbs (Dynabeads, Gibco, #11131D) and transduced 24 h later with the lentivirus by overnight incubation in the presence of polybrene (Santa Cruz, #sc-134220) at 8 μg/ml. A period of cell expansion of 6-8 days was necessary before conducting experiments. Three different cell transductions using three different PBMC donors were used to conduct the experiments in triplicate.

### Example 1.1.4. Flow Cytometry

The following MAbs against human proteins, all from BD Biosciences, were used: CD3-FITC, CD4-BV421, CD8-APC, CD19-PE, and CD33-PE. 7-AAD was used as a viability marker (ThermoFisher, #A1310). CAR19 expression was detected with an APC-conjugated AffiniPureF(ab′)2-fragment goat-anti-mouse IgG (Jackson Immunoresearch. Cat. N. 115-136-072). Samples were run through the fluorescence-activated cell sorting flow cytometer BD FACSCanto II (BD Biosciences) and data were analyzed using the BD FACSDiva Software.

### Example 1.1.5. In Vitro Assays of Anti-Tumor Efficacy

CART19 or untransduced T (UT) cells were co-cultured for 16 h, unless otherwise indicated, with tumor target cell lines (NALM6 or HL60) or primary B-ALL tumor cells, at different effector to target (E:T) ratios, maintaining a fixed number of target cells. Then, cells were transferred to TruCOUNT tubes (BD, Cat. N. 340334) and incubated with MAbs against human CD4, CD8, CD19 (or CD33) and 7-AAD. Cytotoxicity was determined by calculating the number of surviving target cells (identified as 7-AAD negative/CD19 or CD33 positive cells, for NALM6 and HL60 target cells, respectively). Acquisition was stopped after a fixed number of beads was acquired and absolute cell numbers were calculated, allowing comparison between different E:T ratios. Co-culture supernatants were analyzed for cytokine production (IFNγ, TNFα and IL-10) by ELISA, following manufacturer's instructions (BD OptEIA). All experiments were run in triplicate.

CAR cell proliferation in response to CD19 antigen was measured using CFSE assay. Briefly, CAR cells were labeled with 1 μM CFSE, washed and cultured with or without proliferating stimuli (IL-2 50 U/ml, NALM6 or K562 cells). NALM6 or K562 were added at an E:T ratio=1:1. Assay was stopped after 96 h and cells were stained with anti-CD4 and anti-CD8. CFSE staining was measured in CD4+ and CD8+ cells, and proliferating index (PI) was calculated (PI=Sum of number of cells in different generations/calculated number of original cells).

### Example 1.1.6. In Vivo Xenograft Model of Anti-Tumor Efficacy and Safety

Animal studies were approved by CEEA-UB, the competent ethics committee for animal experimentation.

Three-month old NOD.Cg-Prkde^{scid} Il2rd^{tm1Wijl}/SzJ (NSG) mice were infused intravenously (tail vein) with NALM6 tumor cells (1×10⁶ cells/mice) expressing green-fluorescent protein (GFP) and luciferase. Mice were then randomly allocated to either CAR cells (10×10⁶/mice), UT cells (10×10⁶ cells/mice) or vehicle.

CAR or UT cells were infused three days after the infusion of NALM6. Tumor growth was evaluated weekly by bioluminiscence imaging (Hamamatsu detector) after the intravenous administration of D-luciferin. Mice were sacrificed at days 16-17 and tumor burden was measured in blood and bone marrow samples by flow cytometry.

### Example 1.1.7. Patient-Scale CAR Cell Production

Leukocytapheresis from healthy donors were obtained in the Apheresis Unit at the Hospital Clínic de Barcelona with informed consent and approved by Ethics Committee of our hospital. Apheresis procedures were performed using the Amicus device (Fresenius Kabi, Lake Zurich, IL). A minimum of 1×10⁸ T cells diluted in 50 ml of plasma was required. Cells were cultured in the CliniMACS Prodigy® system (Miltenyi Biotec) using TexMACS® media supplemented with 3% Human AB serum and with IL-7, IL-15 (Miltenyi Biotec #170-076-111 and #170-076-114, respectively). For T cell activation, TransACT GMP Grade (Miltenyi Biotec, Cat. N. 170-076-156) was used.

### Example 1.2. Results

### Example 1.2.1. Validation of Anti-hCD19 Monoclonal Antibody of the Invention

Anti-hCD19 monoclonal antibody of the invention reacts against the mouse lymphoma cell line 300.19 transfected with hCD19, but not with untransfected cells. Anti-hCD19 monoclonal antibody of the invention also reacts with a subset of human peripheral blood cells, as expected. Anti-hCD19 monoclonal antibody reacts with B-cell lines Raji and Daudi, while no reactivity is observed when T, myeloid or NK cell lines are used, consistent with the pattern of CD19 expression. Furthermore, we show that pre-incubation of Daudi cells with the anti-CD19 FMC63 antibody, blocks the binding of the monoclonal antibody, confirming its specificity for CD19. Finally, a band of around 100 KDa was precipitated from Daudi cells using anti-hCD19 monoclonal antibody, consistent with the expected molecular weight of CD19. All this data together indicates that the monoclonal antibody of the invention is a highly sensitive and specific antibody for human CD19 protein.

### Example 1.2.2. In Vitro Evaluation of CAR Efficacy

ScFv of anti-CD19 antibody of the invention was cloned in frame with the rest of the CAR signaling domains in a lentiviral vector (pCCL). For evaluation of CAR19 efficacy, PBMCs isolated from buffy coats were activated using CD3 and CD28 dynabeads and subsequently transduced using CAR19-containing lentivirus. After an expansion period, expression of CAR19 on T cells was confirmed by Flow Cytometry. The percentage of CAR cells varied between 20 to 56% depending on the experiment.

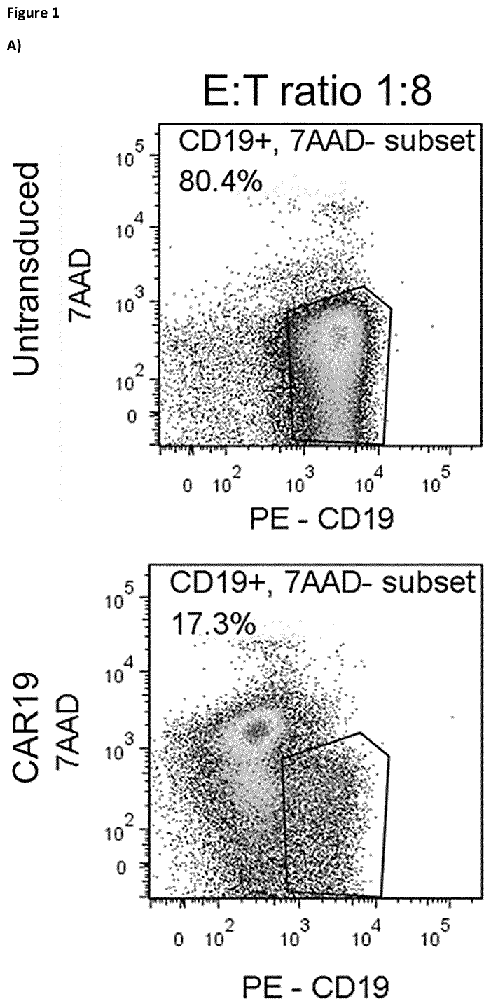

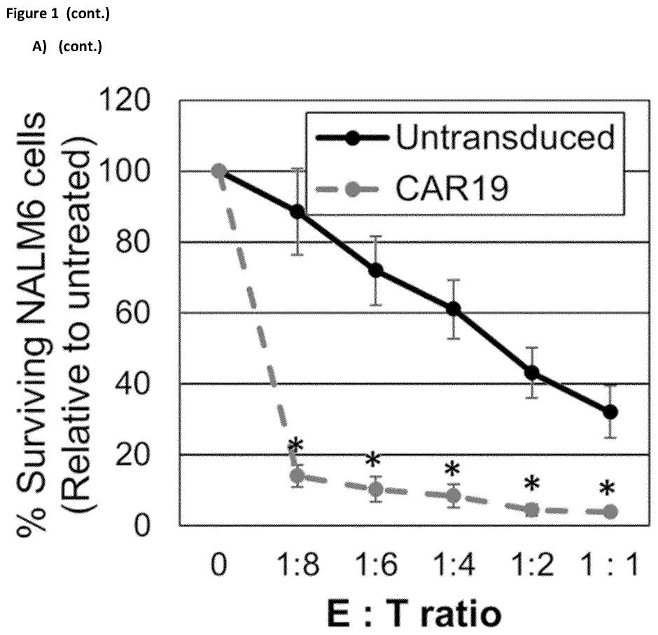

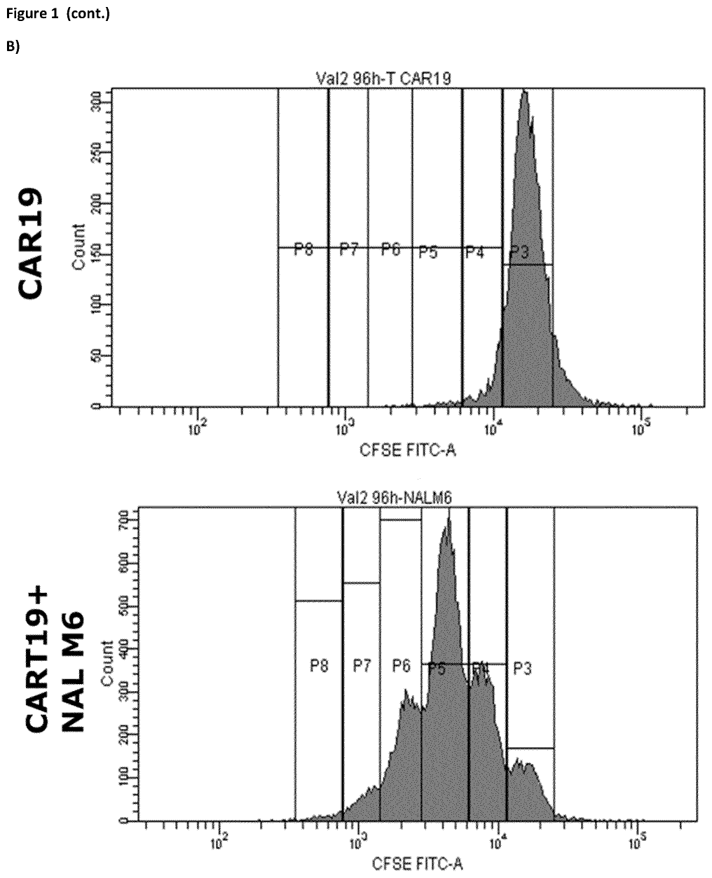

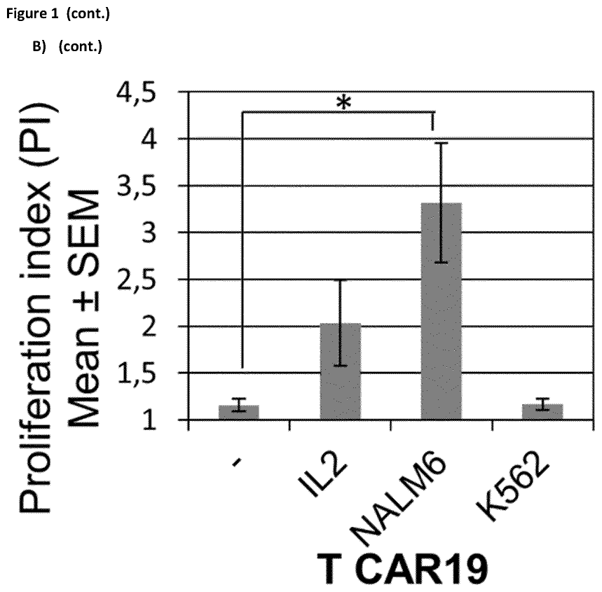

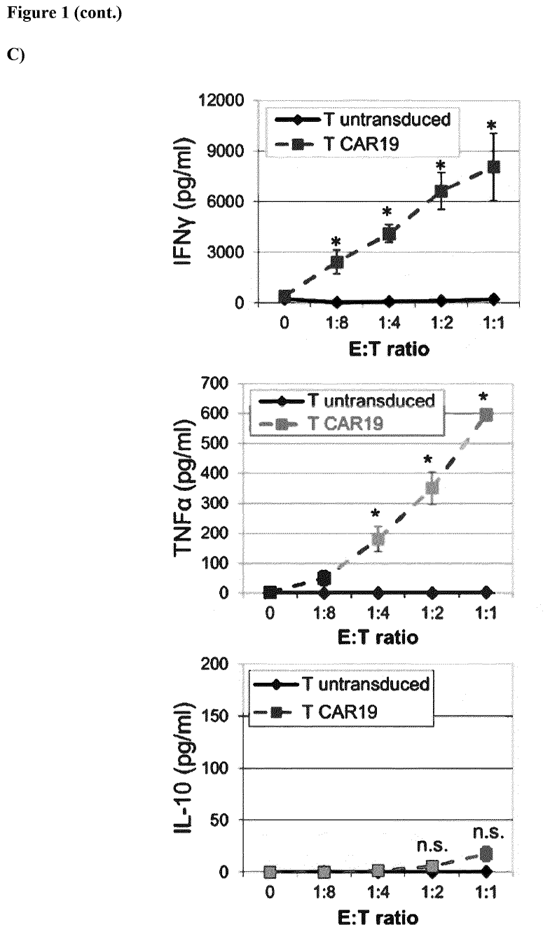

Cytotoxicity of CAR cells was measured by the in vitro eradication of the CD19 positive NALM6 cell line. For this purpose, we developed a flow cytometry-based assay to quantify the number of viable, CD19+ cells (see Material and methods section). NALM6 cells were almost completely eliminated after 16 h of co-culture even after very low E:T ratios (1 effector cell for every 8 target cells). We also observed a minor cytotoxic effect of untransduced (UT) cells due to alloreactivity (FIG. 1A). Target cell specificity was also tested by measuring survival of a CD19 negative HL60 cell line in co-culture with CAR cells.

As expected, no CAR-mediated killing was appreciated in this case. The cytotoxicity of CAR cells was also tested against primary B-ALL cells, demonstrating similar efficacy. All this data together indicate that our CAR cells exhibit a potent and specific cytotoxic effect against CD19 positive cells in vitro.

To better characterize CAR cell response upon CD19 binding, cell proliferation was measured by using a standard CFSE assay at 96 h time-point. Antigen binding should be able to promote CAR cell expansion in order to eliminate a tumor in vivo. As shown in FIG. 1B, CAR cells proliferated in contact with the CD19+ NALM6 cell line and in response to IL-2 (to a minor extent). No proliferation was observed in the absence of stimulus or in contact with a CD19 negative cell line (K562), confirming that cell proliferation was mediated by CD19 recognition.

Finally, CAR cell production of cytokines was measured in the supernatant of effector-target cell co-cultures after 16 h and analyzed using an ELISA assay. Cytokine levels from co-cultures using CAR or UT cells were compared (FIG. 1C). While UT cells did not show an increase in IFNγ and TNFα, CAR cells showed a significant increase in these two pro-inflammatory cytokines. As expected, a very minor and not significant increase in the anti-inflammatory cytokine IL-10 was observed.

### Example 1.2.3. Comparison of Cytotoxic Activity of CAR Cells if the Invention to Other CART19 Constructs

To investigate how CAR cells, which contain the scFv from the antibody of the invention, perform compared to other CART19 cells currently in use for the treatment of CD19+ malignancies, we cloned the scFv of the FMC63 antibody in our vector. The rest of the CAR construct remained the same, so we could directly compare efficiency of both scFv fragments. For these analyses, we transduced PBMCs with a lentivirus containing each one of the CAR constructs. CAR19 protein expression was confirmed by Western Blot. Cytotoxic activity of the antibody of the invention and FMC63 CART cells was then compared in vitro. No significant difference in cytotoxic potency between both CARs, indicating that scFv of the antibody of the invention is equally capable of binding to CD19 and triggering a cytotoxic response.

### Example 1.2.4. In Vivo Evaluation of CAR Efficacy

In order to evaluate the efficacy of the CART19 cells in vivo, we performed a xenograft experiment in NSG mice.

Mice were randomly allocated to the administration of vehicle (A), UT cells (B), CAR cells (C), NALM6 cells (D), NALM6 plus UT cells (E), and NALM6 plus CAR cells (F). Mice corresponding to groups D, E and F were inoculated with NALM6-Luc+GFP+ (CD19+) cells through their tail vein on day 1. On day 4, mice belonging to groups B, C, E and F were infused with either UT cells or CAR cells.

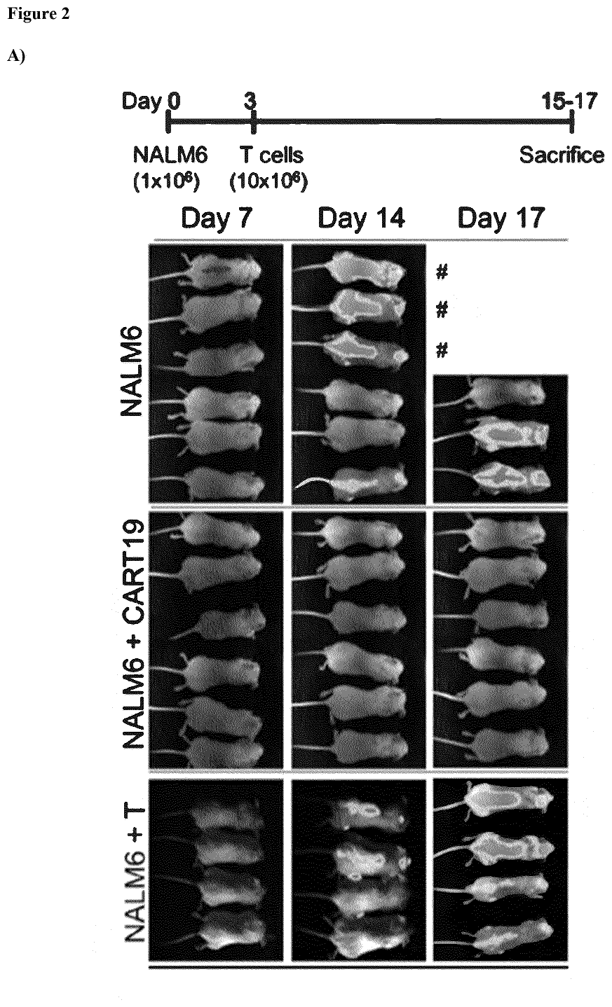

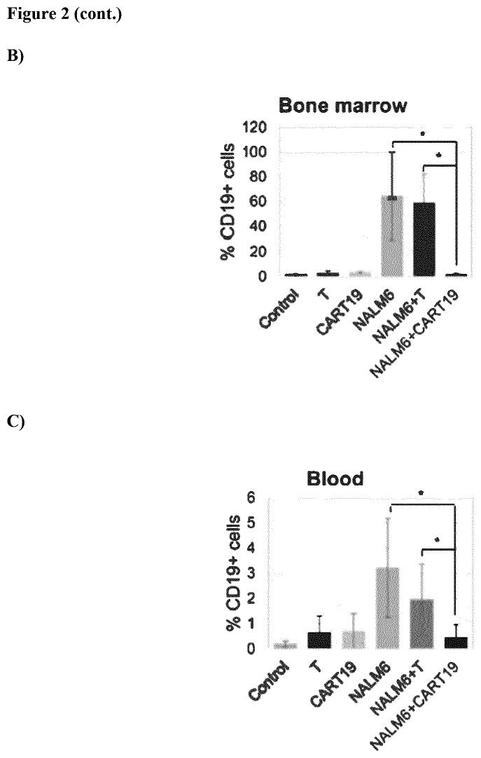

As shown in FIG. 2A, disease progression was clearly observed during a period of 2 weeks following NALM6 cell infusion in NALM6 group (5 out of 6 animals progressed) and NALM6+UT cells group (4 out of 4 animals progressed). However, no disease was detected in any of the mice belonging to the NALM6+CAR group (0 out of 6 mice progressed). The experiment was completed, and the animals sacrificed at days 16-17, when the animals belonging to the NALM6 group started to show evident signs of disease. To confirm bioluminescent imaging data, bone marrow and blood cells were processed for flow cytometry. Anti-human CD19 staining confirmed the presence of tumor cells in NALM6 and NALM6+UT groups, while no significant percentages of tumor cells were detected in the other groups (FIG. 2B-2C) compared to control.

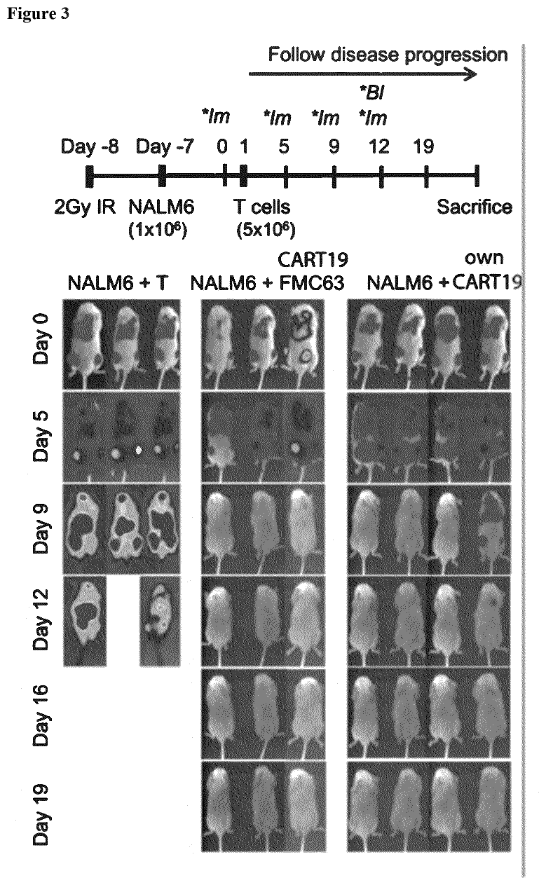

Also FIG. 3 shows and comparison of antitumor activity of the CART cells of the invention with FMC63-based CART cells. Upper panel shows a timeline of experimental design (*Im, bioluminescent image; *BI, blood sample). Lower panels show bioluminescence images showing disease progression at different days.

### Example 1.2.5. Large-Scale CAR19 Lentivirus Production

Having demonstrated the efficacy and specificity of CAR cells in vivo and in vitro, we proceeded to set up and standardize the conditions for patient-scale CAR cell production.

To produce enough lentiviral supernatant to complete the clinical trial, we scaled-up our virus production method and conducted the entire process inside a clean room facility following GMP guidelines, although lentiviral supernatant was considered to be an intermediate reagent in terms of drug agency approval. Each lot consisted of 4 L of unconcentrated virus and the production time/lot was 12 days. HEK293T was used as packaging cell line. Before starting production, HEK293T master cell bank and working cell bank was prepared, so all lots were produced using HEK293T from the same passage. For each production, we first expanded HEK293T in T175 flasks for 2 passages (expanding from 80×10⁶ cells to a minimum of 2829×10⁶ cells). Cells were then transferred to four 10-layer CellStacks cell culture chambers (Corning) and one 1-layer CellStack to control for cell proliferation. Plasmid transfection was carried out the next day using 3.86 mg of PEI, 763 μg Transfer vector, 377 μg pMDLg/pRRE, 188 μg pRSV-Rev and 221 μg pMD2.G per liter. Viral supernatants were collected 2 days later and clarified using a 0.45 μm PVDF membrane. 4 L of viral supernatant were finally concentrated and diafiltered using KrosFlo Research IIi Tangential Flow Filtration System® (Spectrum Labs) and 500 kD mPES hollow fibers. 2 L of PBS was used as diafiltration buffer. Each lot was concentrated to 100 ml, aliquoted in 10 ml bags and kept at −80° C. until use. Smaller aliquots were also kept for viral titer determination, sterility and purity analyses. For protocol validation, 3 viral lots were produced and analyzed. The results of analyses performed on these 3 lots are shown in Table 1. Viral titer of frozen-concentrated virus ranged between 1.1-2.2×10⁸ TU/ml. Quality control testing indicated that all three lots were negative for bacterial-fungal growth, mycoplasma or replication-competent lentivirus (RCL). Virus identity was also confirmed by PCR amplification of principal virus components.

| Parameter | Method | Acceptance Criteria | Lot 1 | Lot 2 | Lot 3 |
|---|---|---|---|---|---|
| Appearance | visual inspection | yellowish | cloudy | cloudy | cloudy |
| | | liquid solution | liquid solution | liquid solution | liquid solution |
| Viral titer | limiting dilution | >3.75 × 10⁷ TU/mL | 2.29 × 10⁸ TU/mL | 1.68 × 10⁸ TU/mL | 1.10 × 10⁸ TU/mL |
| Sterility | microbial growth | sterile | sterile | sterile | sterile |
| Mycoplasma | PCR | absent | absent | absent | absent |
| Identity | PCR | positive | positive | positive | positive |
| RCL (replication | real-time PCR | absent | absent | absent | absent |
| competent lentivirus) | | | | | |

### Example 1.2.6. CAR Cell Production

CAR cells were produced using CliniMACS Prodigy (Miltenyi). Apheresis products were subjected to CD4 and CD8 positive selection and 100×10⁶ T-cells were then cultured and activated using anti-CD3 and anti-CD28 antibodies. 24 h after activation, cells were transduced with CAR19 lentivirus at a MOI=10. Cells were cultured in media containing IL-7 and IL-15 until the desired cell number was reached (typically 8-9 days). The product was collected in NaCl 0.9%+0.5% HSA. To test the consistency and robustness of our production method, we conducted three procedures using apheresis products from three different healthy donors. Our goal was to reach a minimum of 35×10⁶ CAR cells and ≥20% transduction efficiency.

The expansion time varied between 8 and 11 days. Run was allowed to proceed to day 11 to test Prodigy's T cell expansion capacity but the rest of the runs were stopped earlier (day 9 and day 8 respectively) since the minimum number of cells required was already reached. A mean of 3780×10⁶ total cells were obtained, and the percentage of transduction averaged 35.8% at the time the cell expansion was terminated. Therefore, the acceptance criteria were reached in all three procedures. A full list of the quality tests conducted on the final products and the acceptance criteria that were defined for each of them is provided in Table 2. As shown in the same table, all CAR products obtained met the acceptance criteria established for all parameters regarding purity, safety and potency.

| Parameter | Method | Acceptance Criteria | ARI0001/01 | ARI0001/02 | ARI0001/03 |
|---|---|---|---|---|---|
| Appearance | visual inspection | cloudy liquid solution | cloudy liquid | cloudy liquid | cloudy liquid |
| | | | solution | solution | solution |
| Number of | Neubauer cell counting | 35 × 10⁶ cells for a 70-kg patient | 1,495 × 10⁶ | 1,746 × 10⁶ | 1,340 × 10⁶ |
| CAR19+ cells: | and flow cytometry | (>0.5 × 10⁶ cells/kg) | | | |
| CAR19+ cells (%) | flow cytometry | ≥20% | 26.0 | 41.9 | 39.7 |
| CD3+ cells (%) | flow cytometry | ≥70% | 97.9 | 98.6 | 98.6 |
| Cell viability (%) | Neubauer cell counting | ≥70% | 97.9 | 98.1 | 98.0 |
| | with trypan blue exclusion | | | | |
| Sterility | microbial growth | sterile | sterile | sterile | sterile |
| Mycoplasma | PCR | absent | absent | absent | absent |
| Endotoxin | chromogenic assay | ≤0.5 EU/mL | ≤0.5 EU/mL | ≤0.5 EU/mL | ≤0.5 EU/mL |
| Adventitious viruses | PCR | absent | absent | absent | absent |
| Number of transgene | real-time PCR | ≤10 copies/cell | 1.45 | 2.32 | 2.51 |
| copies/cell | | | | | |
| RCL (replication- | real-time PCR | absent | absent | absent | absent |
| competent | | | | | |
| lentivirus) | | | | | |
| Cytotoxic potency | flow cytometry | (a) NALM6 cell surviving fraction | (a) 14.9; | (a) 40.4; | (a) 43.0; |
| | | using CART19 ratio E:T 1:1 <70% | (b) 90.9 | (b) 64.6 | (b) 67.0 |
| | | or (b) difference in NALM6 cell | | | |
| | | surviving fraction between CART19 | | | |
| | | versus untransduced T using ratio | | | |
| | | E:T 4:1 >50% | | | |

### Example 2. Car T-Cell Production

### Example 2.1. Material and Methods

### Example 2.1.1. Patients and Samples

At the time of submitting this manuscript, 28 products from 27 patients enrolled in phase I clinical trial for CD19+ B-cell malignancies have been produced. Among the 27 patients, 22 had ALL (14 adult and 8 pediatric patients), 4 had NHL and 1 CLL. All patients included in the clinical trial had relapsed of their disease. Patients' pretreatment regimens are summarized in Table 3.

TABLE 3

| Pat ID | Age | Sex | Disease | Lines of treatment prior to leukoapheresis | Allogeneic HCT |
|---|---|---|---|---|---|
| T01 | 27 | M | ALL | PETHEMA 2011, blinatumomab, radiotherapy | Yes (+DLI) |
| T02 | 25 | M | PMLBCL | R-CHOP, R-ESHAP, autologous HCT, radiotherapy, brentuximab, | No |
| | | | | GSK525762, nivolumab | |
| T03 | 7 | F | ALL | SEHOP 2013, SEHOP 2016, inotuzumab, methotrexate + | Yes (+DLI) |
| | | | | cyclophosphamide + anthracyclins + prednisone | |
| T04 | 19 | F | PMLBCL | R-CHOP R-ESHAP, autologous HCT, radiotherapy, | No |
| | | | | BURKIMAB, Gemcitabine + vinorelbine + procarbacine | |
| T05 | 51 | M | DLBCL | BURKIMAB, autologous HCT, cyclophosphamide + prednisone, | No |
| | | | | GSK525762, methotrexate, cyclophosphamide | |
| T06 | 20 | F | ALL | PETHEMA 2011, PETHEMA 2008 | Yes |
| T07 | 19 | M | ALL | PETHEMA 2011, FLAG-Ida | Yes |
| T08 | 53 | F | CLL | FCR, BR, ibrutinib, venetoclax, obinutuzumab, idelalisib | No |
| T09 | 8 | M | ALL | SEHOP 2008, SEHOP 2013 | No |
| T13 | 20 | M | ALL | GRAAL 2003, FLAG-Ida, blinatumomab | Yes (x2) |
| T11 | 34 | F | ALL | PETHEMA 2011, Hyper-CVAD, inotuzumab | Yes |
| T12 | 3 | F | ALL | SEHOP 2013, SEHOP 2016, vincristine + prednisone | Yes |
| T14 | 27 | M | ALL | PETHEMA 2008, FLAG-Ida, PETHEMA 2011 | Yes |
| T15 | 30 | M | ALL | PETHEMA 2011, FLAG-Ida, FLAG-Ida + blinatumomab, FLAG-Ida, inotuzumab | Yes (x2 + DLI) |
| T16 | 10 | M | ALL | SEHOP 2013 | Yes |
| T17 | 23 | M | ALL | PETHEMA 2011, FLAG-Ida, PETHEMA 2008, BFM-90, inotuzumab | Yes |
| T19 | 9 | F | ALL | PETHEMA, SEHOP 2015, radiotherapy | No |
| T20 | 35 | M | ALL | PETHEMA 2011, FLAG-Ida, PETHEMA 2011, methotrexate + | Yes |
| | | | | vincristine + dexamethasone | |
| T21 | 13 | F | ALL | SEHOP 2013, SEHOP 2016 prednisone | Yes |
| T22 | 29 | M | ALL | PETHEMA 2008, dasatinib, FLAG-Ida + ponatinib, FCR | Yes |
| T24 | 19 | M | ALL | PETHEMA 2011, FLAG-Ida | No |
| T25 | 47 | F | ALL | PETHEMA 2011, FLAG-Ida, PETHEMA 2011 | Yes |
| T26 | 8 | F | ALL | SEHOP | Yes |
| T27 | 22 | M | ALL | PETHEMA 2008, FLAG-Ida, PETHEMA 2011 | No |
| T30 | 31 | M | ALL | PETHEMA 2008, blinatumomab | Yes (+DLI) |
| T32 | 23 | M | ALL | PETHEMA 2008, PETHEMA 2011, vindesine + prednisone, inotuzumab | Yes (x2) |
| T34 | 45 | F | DLBCL | R-CHOP R-ESHAP BURKIMAB, radiotherapy | No |

Abbreviations:
ALL, acute lymphoblastic leukemia;
DLBCL, diffuse large B-cell lymphoma;
PMLBCL, primary mediastinal large B-cell lymphoma;
CLL, chronic lymphocytic leukemia;
DLI, donor lymphocyte infusion;
HCT, hematopoietic cell transplantation;
FCR, fludarabina + cyclophosphamide + rituximab;
BR, bendamustina + rituximab;
FLAG-Ida, fludarabina + cytarabine + idarubicin + G-CSF;
PETHEMA, Spanish Program of Treatments in Hematology;
SEHOP, Spanish Society of Pediatric Hematology & Oncology;
GRAAL, Group for Research on Adult Acute Lymphoblastic Leukemia

Adult patients were subjected to leukocytapheresis at the Apheresis Unit, Hospital Clínic, and pediatric patients at the Apheresis Unit of Hospital Sant Joan de Déu/BST, after signing an informed consent. Apheresis procedures were performed using Amicus device (Fresenius Kabi, Lake Zurich, IL). A minimum of 1×10⁸ total T-cells diluted in 50 ml of plasma were required. This study has been approved by the Research Ethics Comitee (Celm) of Hospital Clinic. HCB/2017/0001. Clinical trial: CART19-BE-01. Eudra: 2016-002972-29.

### Example 2.1.2. Production of CAR Cells of the Invention

Apheresis products were connected to CliniMACS Prodigy® system (Miltenyi Biotec) tubing set. Erythrocytes and platelets were removed by density gradient centrifugation in the Centricult unit. The remaining cells were selected using CD4 and CD8 coated magnetic beads. Selected cells were eluted in the “Reapplication Bag”. After selection, 1×10⁸ T-cells (from reapplication bag) were used to initiate cell culture. The remaining cells were cryopreserved in bags and vials to be used as control cells for product quality assays and as a backup in case of production failure. Cells were cultured using TexMACS® media supplemented with 3% human AB serum (obtained from the blood bank. BST) and with 155 IU/mL IL-7 and 290 IU/mL IL-15 (Miltenyi Biotec #170-076-111 and #170-076-114, respectively). Cells were immediately activated using TransACT GMP Grade (Miltenyi Biotec, Cat. N. 170-076-156) and transduced 24 h later using CAR19-containing lentivirus at MOI=10. A cell culture wash was programmed 48 h after transduction. The cells were then maintained in culture with increasing shaking until the desired cell number was reached (typically 7-10 days after cell culture initiation). Cells were finally eluted in 100 ml 0.9% NaCl+1% HSA, aliquoted according to the desired dose of CAR cells of the invention and cryopreserved until infusion.

The aim was to achieve 2 doses of CAR cells of the invention cells/patient. The planned target cell dose varied depending on the patient's disease. Typically, 1×10⁶ CAR cells of the invention (cells/kg) for patients with ALL and CLL, and 5×10⁶ of CAR cells of the invention (cells/kg) for NHL patients.

### Example 2.1.3. Monoclonal Antibodies

CAR19 expression was detected with an APC-conjugated AffiniPureF(ab′)2-fragment goat-anti-mouse IgG (Jackson ImmunoResearch Laboratories, 115-136-072). The product composition comprising the CAR cells of the invention was determined by flow cytometry using staining with the following antibodies (all from BD): CD45-APC, CD3-BV421, CD4-FITC, CD8-PerCPCy5.5, CD19-PECy7, CD16-PE, CD56-PE.

For the T cell subset characterization experiments, CAR+ cells were detected using a CD19-Fc recombinant protein chimera (R&D, Cat. N. 9269-CD-050) and a secondary antibody FITC-Goat F(ab)2 anti-human IgG (Life Technologies, Cat. N. H10101C). This staining was combined with the following monoclonal antibodies (all from BD): CD3-BV421, CD8-APC.Cy7, CD45RA-PECy7, CD45RO-APC, CCR7-PerCPCy5.5, CD28-BV510 and CD95-PE (or CD27-PE). T cell subpopulations were defined as follows: T_{N}: CD45RA+, CCR7+; T_{SCM}: CD45RA+, CCR7+, CD95+; T_{CM}: CD45RA−, CCR7+; T_{EM}: CD45RA−, CCR7− and T_{EFF}: CD45RA+, CCR7−.

For intracellular cytokine measurement, the following antibodies were used, all from BD: CD3-BV450, CD8-APC.H7, CD4-BV500, IFNγ-PerCP.Cy5.5, TNFα-PE.

For repeated challenges experiment, the antibodies used were the following, all from BD: CD3-APC, CD4-BV510, CD8-APC.Cy7, CD19-PE.

For flow cytometry analyses, cells were acquired using a FACS Canto II, BD and subsequently analyzed using FlowJo Software.

### Example 2.1.4. Product Quality Controls

Product potency assay was performed by flow cytometry. Real-time PCR was used to measure number of copies/cell and to assess the presence of replication-competent lentivirus (RCL) in the final product. Product sterility, absence of mycoplasma, endotoxin and adventitious virus was determined by a certified laboratory. Adventitious virus included the determination of HIV virus presence among others. Since conventional HIV detection methods detect also the presence of the lentiviral transgene used to transduce the cells, an alternative PCR assay based on the detection of Env gene was used to discriminate between HIV infection and lentiviral transduction.

### Example 2.1.5. Cytokine Measurement

Cytokine level was measured using Milliplex MAP Human Cytokine/Chemokine Magnetic Bead panels (Millipore). A 10-plex kit for IFNγ, IL-10, IL-1B, IL-6, TNFα, IL-12 (P40), IL-17, IL-2, IL-4 and IP-10, a 3-plex kit for IL-8, IL-15 and MIP1A (Cat N. HCYTOMAG-60K) and a 1-plex kit for GranzymeB (Cat. N. HCD8MAG-15K) were used. The assay was performed following manufacturer's instructions. Samples were run in a Luminex 200 system.

Alternatively, intracellular cytokine production (IFNγ and TNFα) was measured by flow cytometry. Briefly, cells were first labeled for extracellular markers CD4, CD8 and CD3 and incubated 15 min. Cells were then fixed using 1×BD lysing solution (Cat. N. 349202) and incubated for an additional 15 min. After 2 washes, cells were permeabilized using FACS buffer+0.1% saponin, and incubated for 15 min. Cells were then incubated with anti-IFNγ and anti-TNFα, for 30 min at 4° C. After that, cells were washed in PBS and analyzed.

### Example 2.1.6. Small-Scale T Cell Expansions

0.5×10⁶ T-cells were cultured with X-Vivo 15 Cell Medium (Cultek, Cat. N. BE02-060Q), 5% AB human serum (Sigma, Cat. N. H4522), penicillin-streptomycin (100 μg/ml) and the indicated cytokine: 50 IU/ml IL-2 (Miltenyi Biotec) or 155 IU/mL IL-7 and 290 IU/mL IL-15 (Miltenyi Biotec). Cytokines were added to the media every 48 h. 24 h after thawing cells were activated with Dynabeads Human T-Activator CD3/CD28 (Gibco, Cat. N. 11131D) according to the manufacturer's instructions. Cells were transduced after an additional 24 h with a MOI of 10 and then expanded for 11 days at a concentration of 0.5 ×10⁶ to 1.5×10⁶ T-cells/ml.

### Example 2.1.7. T-Cell Expansion after Repeated Challenges with Target Cells

To analyze T-cell proliferation capacity after antigen encounter, we seeded a co-culture of CAR-T cells and NALM6 cells at 1:1 ratio (250.000 cells each). After 4 days of incubation, an aliquot of the culture was taken and analyzed to determine T-cell number. Cells were labeled with CD3, CD4, CD8 and CD19, and then 20 μl of beads (CountBright, Cat. N. C36950, Invitrogen) was added to the sample to determine absolute cell number. This process was repeated 3 times.

### Example 2.1.8. Statistics

Statistical significance was assessed using SPSS software. Unpaired T-test was used unless otherwise specified. U-Mann Whitney was used for comparison of variables with non-normal distributions. Statistical significance was considered when p value ≤0.05.

### Example 2.2. Results

### Example 2.2.1. CAR T-Cell Expansion

28 apheresis products were obtained from 27 patients included in the clinical trial. For one patient, the apheresis product was obtained twice due to CAR cells of the invention production failure (T10 and T13 products belong to the same patient). Description of apheresis products is presented in Table 4. Patients' apheresis products were subjected to CD4+ and CD8+ magnetic selection using the CliniMACS Prodigy system. In all cases except for one (Patient T27), the minimum number of T-cells (100×10⁶) was obtained (Table 4). In Patient T27, cell culture was initiated with 50×10⁶ cells.

TABLE 4

| | | | | | | | CD4-CD8 |
|---|---|---|---|---|---|---|---|
| | | | | | | | selection |
| | | | | | | | Total |
| | | | | | | | cell |
| Pat. | | Apheresis products | | | | | number |
| ID | Disease | WBC (×10⁹) | Lymph (×10⁹) | CD3+ (%)* | CD4+ (%)* | CD8+ (%)* | (×10⁶) |
| T01 | Adult ALL | 7.15 | 6.40 | 24.80 | 12.60 | 10.70 | 960 |
| T02 | NHL | 3.31 | 1.56 | 32.50 | 11.40 | 19.80 | 1,200 |
| T03 | Pediatric ALL | 4.59 | 3.50 | 49.38 | 22.67 | 26.71 | 1,580 |
| T04 | NHL | 4.04 | 0.85 | 13.50 | 6.60 | 6.20 | 488 |
| T05 | NHL | 7.71 | 4.23 | 30.00 | 12.10 | 15.50 | 1,800 |
| T06 | Adult ALL | 3.64 | 3.19 | 28.80 | 10.60 | 16.20 | 720 |
| T07 | Adult ALL | 4.91 | 3.84 | 29.00 | 4.40 | 19.80 | 1,900 |
| T08 | CLL | 2.88 | 2.30 | 19.70 | 8.80 | 10.10 | 1,560 |
| T09 | Pediatric ALL | 4.27 | 3.24 | 21.50 | 11.30 | 8.80 | 1,442 |
| T10 | Adult ALL | 1.00 | 0.72 | 15.30 | 7.90 | 6.10 | 350 |
| T11 | Adult ALL | 2.08 | 0.98 | 13.90 | 7.90 | 4.40 | 360 |
| T12 | Pediatric ALL | 8.40 | 6.21 | 2.20 | 1.70 | 0.50 | 150 |
| T13 | Adult ALL | 1.29 | 0.64 | 26.90 | 8.40 | 15.60 | 250 |
| T14 | Adult ALL | 3.22 | 2.31 | 38.90 | 17.80 | 18.00 | 2,160 |
| T15 | Adult ALL | 2.88 | 1.28 | 26.10 | 8.70 | 14.50 | 1,050 |
| T16 | Pediatric ALL | 1.98 | 1.22 | 22.60 | 4.80 | 15.90 | 400 |
| T17 | Adult ALL | 8.55 | 2.82 | 27.90 | 7.40 | 19.20 | 1,400 |
| T19 | Pediatric ALL | 1.09 | 0.20 | 22.50 | 12.80 | 7.50 | 250 |
| T20 | Adult ALL | 3.73 | 2.23 | 28.60 | 7.20 | 20.70 | 760 |
| T21 | Pediatric ALL | 4.50 | 2.20 | 36.20 | 18.60 | 14.20 | 400 |
| T22 | Adult ALL | 8.37 | 3.16 | 42.00 | 21.40 | 19.50 | 750 |
| T24 | Adult ALL | 25.85 | 22.00 | 4.10 | 1.20 | 0.70 | 200 |
| T25 | Adult ALL | 0.83 | 0.46 | 32.50 | 5.50 | 25.70 | 1,520 |
| T26 | Pediatric ALL | 0.84 | 0.51 | 6.50 | 1.40 | 4.60 | 200 |
| T27 | Adult ALL | 0.33 | 0.10 | 10.00 | 3.30 | 6.10 | 50 |
| T30 | Adult ALL | 2.06 | 1.48 | 46.50 | 13.20 | 28.80 | 871 |
| T32 | Adult ALL | 2.23 | 0.69 | 52.00 | 19.30 | 27.40 | 2,000 |
| T34 | NHL | 6.71 | 3.64 | 23.50 | 12.60 | 9.70 | 993 |

*% of cells over WBC

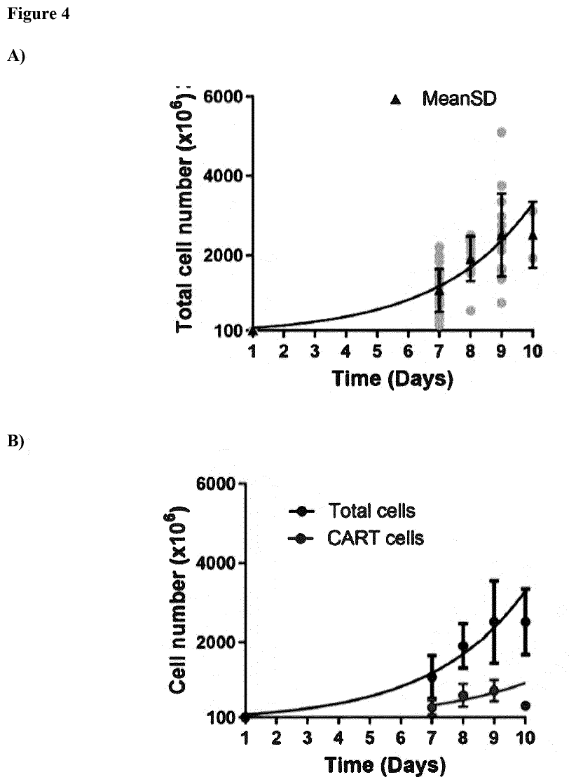

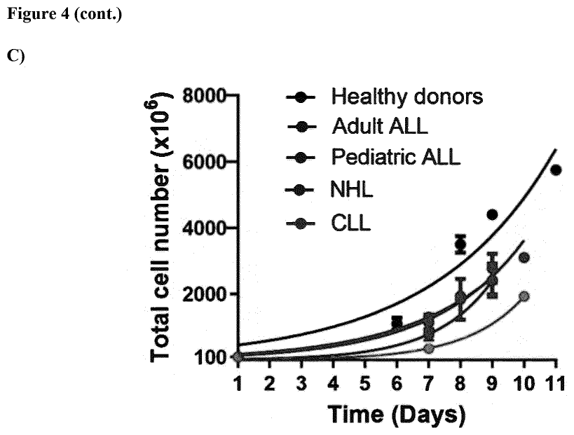

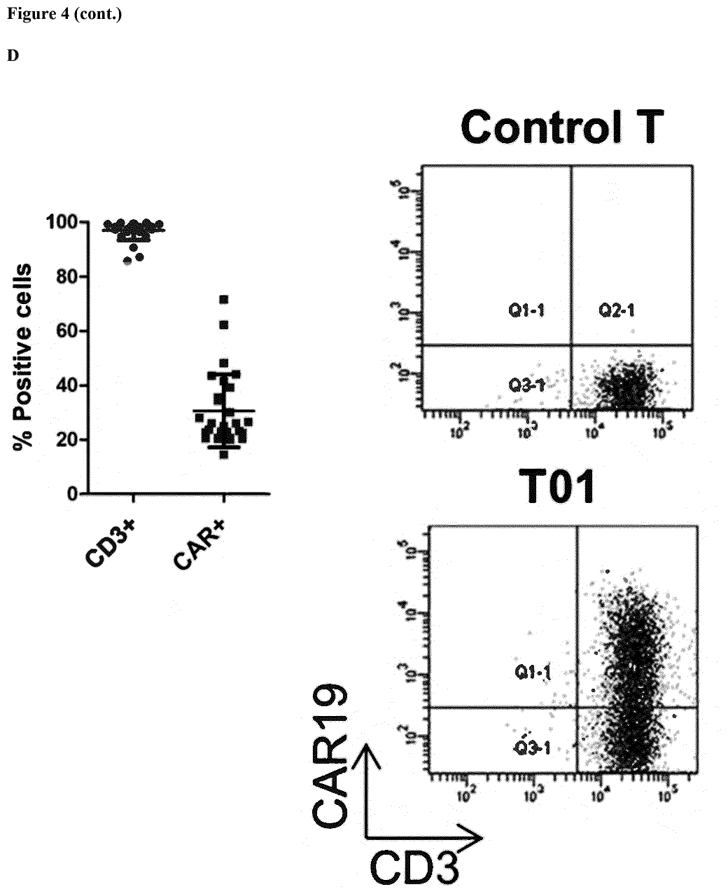

Results of cell expansion in CliniMACS Prodigy for the 27 products are presented in FIG. 4A-B. Cells were expanded for an average of 8.5 days, range 7 to 10. Average total cell number obtained in the final product was 2548×10⁶, range 600×10⁶ to 5200×10⁶. In one patient where cell culture was started with 50×10⁶ cells, the final product also met acceptance criteria. In this particular case, cell culture was maintained for 13 days, finally obtaining 3300×10⁶ cells. When compared to healthy donors (used in 3 previous validation runs), patient cells seem to expand more slowly, even if the number of runs performed with healthy donors is limited (FIG. 4C).

Products were analyzed in terms of appearance, quantity, identity, purity, safety and potency.

### Example 2.2.2. Product Purity and Transduction Efficiency

The final product was characterized in terms of cell viability, percentage of CD3+ cells and percentage of CAR+ cells. This data is summarized in Table 5.

TABLE 5

| Pat. | Cell | % | % | | | | Adventitious | Transgene | | |
|---|---|---|---|---|---|---|---|---|---|---|
| ID | viability | CD3+ | CAR+ | Sterility | Mycoplasma | Endotoxin | virus | copies/cell | RCL | Potency |
| T01 | 100 | 94.7 | 39.3 | Sterile | Absent | <0.5 EU/ml | Absent | 1.05 | Absent | 13.9 |
| T02 | 95 | 96.5 | 28 | Sterile | Absent | <0.5 EU/ml | Absent | 0.92 | Absent | 6.0 |
| T03 | 90 | 96.0 | 30 | Sterile | Absent | <0.5 EU/ml | Absent | 0.97 | Absent | 2.7 |
| T04 | 98 | 98.1 | 22.8 | Sterile | Absent | <0.5 EU/ml | Absent | 0.66 | Absent | 17.2 |
| T05 | 93 | 98.4 | 22.9 | Sterile | Absent | <0.5 EU/ml | Absent | 0.9 | Absent | 41.4 |
| T06 | 96 | 87.1 | 43.6 | Sterile | Absent | <0.5 EU/ml | Absent | 1.55 | Absent | 0.6 |
| T07 | 96 | 97.3 | 20.4 | Sterile | Absent | <0.5 EU/ml | Absent | 0.73 | Absent | 18.6 |
| T08 | 92 | 98.7 | 20.7 | Sterile | Absent | <0.5 EU/ml | Absent | 1.62 | Absent | 21.4 |
| T09 | 100 | 99.1 | 20.6 | Sterile | Absent | <0.5 EU/ml | Absent | 0.77 | Absent | 8.7 |
| T10 | 98 | 98.7 | 14.5* | — | — | — | — | — | — | — |
| T11 | 94 | 99.7 | 25 | Sterile | Absent | <0.5 EU/ml | Absent | 2.31 | Absent | 0.04 |
| T12 | 98 | 99.4 | 20 | Sterile | Absent | <0.5 EU/ml | Absent | 0.43 | Absent | 14.4 |
| T13 | 95 | 99.2 | 20.4 | Sterile | Absent | <0.5 EU/ml | Absent | 0.57 | Absent | 3.5 |
| T14 | 98 | 99.2 | 62.2 | Sterile | Absent | <0.5 EU/ml | Absent | 1.99 | Absent | 0.01 |
| T15 | 93 | 97.5 | 34.4 | Sterile | Absent | <0.5 EU/ml | Absent | 1.98 | Absent | 1.4 |
| T16 | 91 | 98.7 | 26.6 | Sterile | Absent | <0.5 EU/ml | Absent | 1.46 | Absent | 0.1 |
| T17 | 96 | 94.8 | 26 | Sterile | Absent | <0.5 EU/ml | Absent | 1.47 | Absent | 0.2 |
| T19 | 93 | 90.6 | 22.6 | Sterile | Absent | <0.5 EU/ml | Absent | 0.99 | Absent | 0.3 |
| T20 | 94 | 98.3 | 20.8 | Sterile | Absent | <0.5 EU/ml | Absent | 1.15 | Absent | 0.02 |
| T21 | 91 | 99.4 | 48.2 | Sterile | Absent | <0.5 EU/ml | Absent | 1.59 | Absent | 0.0 |
| T22 | 82 | 97.3 | 44.1 | Sterile | Absent | <0.5 EU/ml | Absent | 2.1 | Absent | 0.0 |
| T24 | 97 | 99.7 | 23.8 | Sterile | Absent | <0.5 EU/ml | Absent | 1.55 | Absent | 4.18 |
| T25 | 94 | 98.2 | 41.5 | Sterile | Absent | <0.5 EU/ml | Absent | 2.92 | Absent | 0.06 |
| T26 | 97 | 98.5 | 23 | Sterile | Absent | <0.5 EU/ml | Absent | 0.4 | Absent | 28.0 |
| T27 | 98 | 96.5 | 23.2 | Sterile | Absent | <0.5 EU/ml | Absent | 0.62 | Absent | 4.3 |
| T30 | 95 | 85.7 | 26 | Sterile | Absent | <0.5 EU/ml | Absent | 2.08 | Absent | 0.2 |
| T32 | 97 | 98.2 | 35.6 | Sterile | Absent | <0.5 EU/ml | Absent | 1.93 | Absent | 0.2 |
| T34 | 94 | 99.1 | 71.5 | Sterile | Absent | <0.5 EU/ml | Absent | 2.93 | Absent | 0.02 |

*Value below acceptance criteria

All products met acceptance criteria for cell viability and percentage of CD3+ cells (>70% for both parameters). The lowest value detected for cell viability was 91% and 85.7% for CD3+ cells (FIG. 4D).

To analyze the percentage of CAR+ cells, we first validated our detection method based on the use of an APC-conjugated F(ab′)2 anti-mouse antibody. To this end, we engineered a vector where CAR19 and GFP were co-expressed. The correlation between GFP+APC+ or GFP−APC− cells was of 93.5%, thereby indicating that the detection method had a good sensitivity and specificity.

Using this detection system, we assessed the percentage of CAR+ (CAR cells of the invention) cells in the patients' products. All products except one met the specification of >20% CAR cells of the invention. In one product (T10) only 14.5% CAR cells of the invention were detected. Consequently, this product was considered a production failure. CAR T-cell production was repeated for this patient from a 2^{nd} apheresis (T13). This time, a valid product could be obtained. Mean (+SD) of percentage of CAR+ cells in this series was 30.6±13.44 (FIG. 4B-4D), slightly lower than transduction efficiencies achieved in small-scale expansions (45.3%). No significant differences in efficiency of transduction were observed between healthy donors and patients (35.8% vs 30.6%), or among the different diseases. Percentage of CAR+ cells over time during cell expansion was also investigated. A high degree of variability was detected among patients, with the percentage of CAR+ cells increasing in some patients while decreasing in others. In terms of number of cell doses obtained per patient, considering a standard weight of 70 kg for adults and 25 kg for pediatric patients, a minimum of 2 cell doses were quickly obtained (by day 7) for all ALL patients (dose 1×10⁶ of CAR cells of the invention; cells/kg). For NHL patients (dose 5×10⁶ of CAR cells of the invention; cells/kg), 2 cell doses were obtained for 3 out of 4 patients, by day 9.

Indeed, the number of cell doses obtained for ALL far exceeded the need (9 cell doses for adult patients and 25.4 for pediatric patients), indicating that the time of ex vivo cell expansion could be reduced if necessary, in these groups of patients. For NHL, the average number of CAR cells of the invention doses obtained was 2.5. Only one CLL patient has been produced so far. T-cells from this patient grew slower and required 10 days of expansion, finally obtaining 398×10⁶ CAR cells of the invention.

CAR19 transduction was also assessed in terms of DNA copies/cell. As shown in Table 5 CAR19 was detected in all products, within a range of 0.4 to 2.9 copies/cell (all below the limit considered safe of <10 copies/cell). As expected, a positive correlation between percentage of CAR+ cells and DNA copies/cell was obtained, further validating both techniques.

### Example 2.2.3. Product Potency

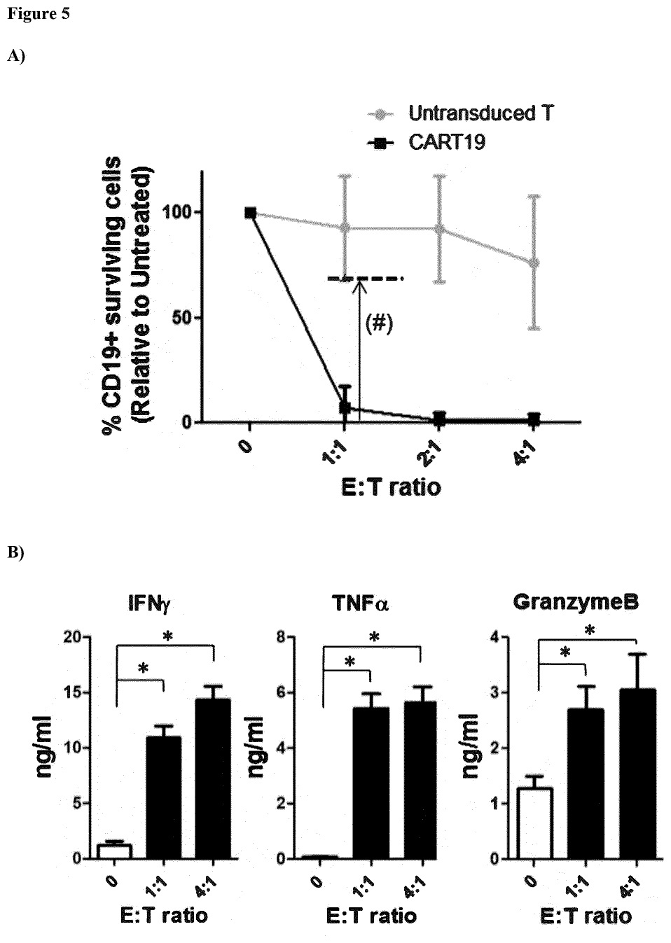

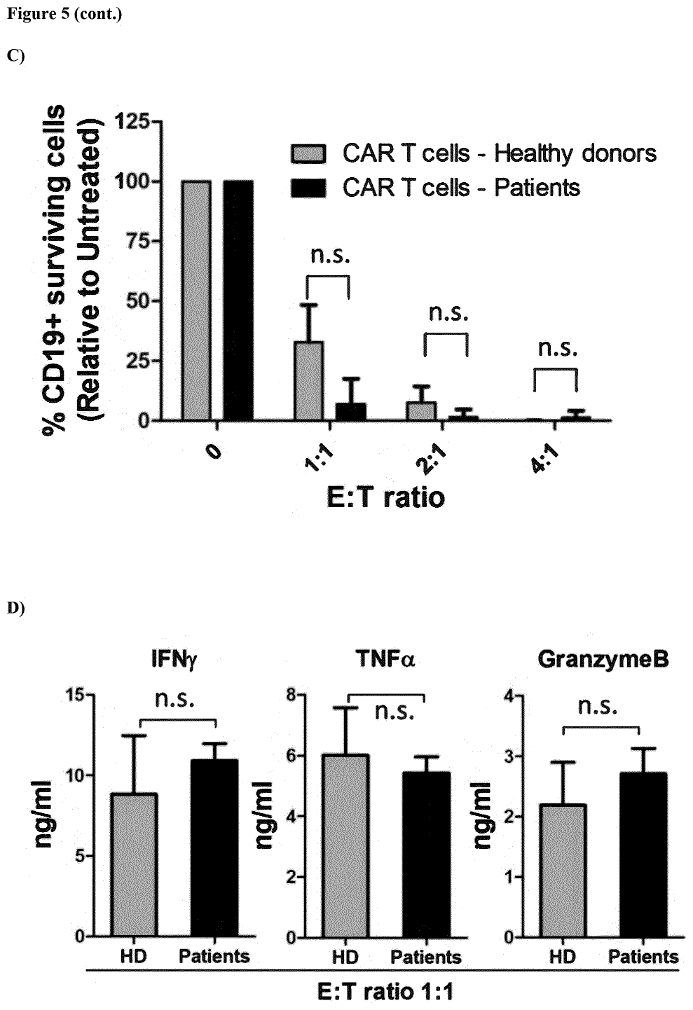

Cytotoxic potential was analyzed in vitro for each product before infusion. A co-culture of the final product with NALM6 cell line was initiated at different E:T ratios. Percentage of alive-CD19+ cells was measured by flow cytometry after 4 h. As a control, the cytotoxic activity of non-transduced CD4+CD8+ cells from the same patient was also measured. Products were considered valid when the CD19+ cell surviving fraction with CAR cells of the invention, at ratio 1:1, was lower than 70%. Results are presented in Table 5 and FIG. 5A. All products obtained met the specification of less than 70% CD19+ surviving fraction at E:T ratio 1:1, indicating that all products prepared had the intrinsic capacity of eliminating CD19+ cells.

Cytokine level was also measured in the supernatant of cytotoxicity assays. As expected, increased levels of pro-inflammatory cytokines such as IFNγ and TNFα was observed when CAR cells of the invention were co-cultured with NALM6, compared to CAR cells of the invention alone. The level of GranzymeB was also significantly increased (FIG. 5B) consistent with the cytotoxic activity of CAR cells of the invention.

CAR T-cells produced from patients were compared to those obtained from healthy controls in terms of cytotoxic activity and cytokine production. As shown in FIG. 5C, patients' and healthy donors' CAR T-cells showed similar cytotoxic potential (even slightly higher for patient's cells although this was not statistically significant). Production of pro-inflammatory cytokines (IFNγ and TNFα) and GranzymeB was also comparable (FIG. 5D).

### Example 2.2.4. T Cell Subset Characterization

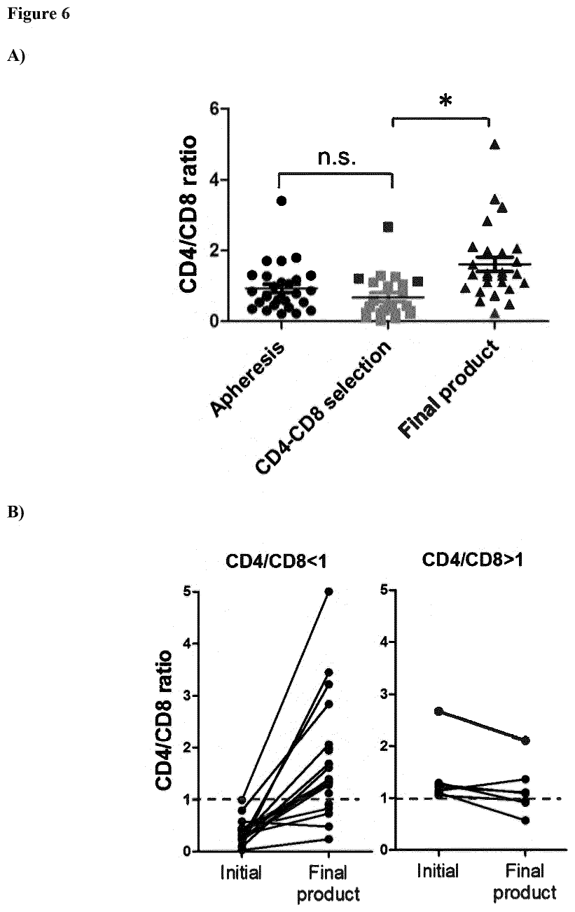

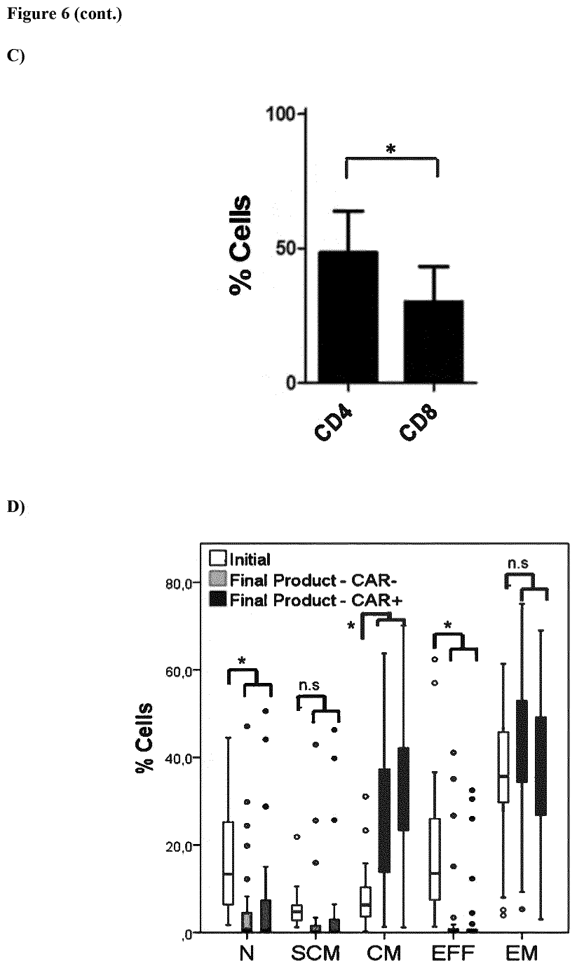

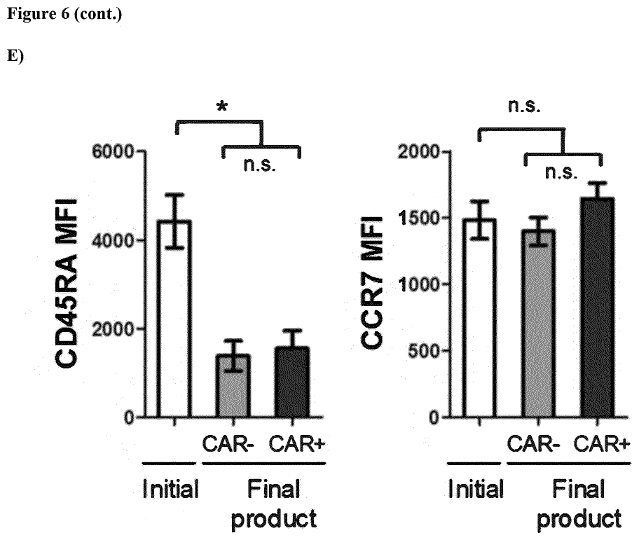

Product composition was further analyzed in terms of CD4/CD8 ratio and T_{N}, T_{SCM}, T_{CM}, T_{E} and T_{EM} subsets. CD4/CD8 ratio was inverted (CD4/CD8 ratio <1) in a large subset of patients that were candidate for a CAR T-cell therapy (FIG. 6A). Average CD4/CD8 ratio was 0.93±0.67 in the apheresis products. This ratio was not significantly altered after CD4 and CD8 cell selection in the vast majority of patients. However, a significant increase in the proportion of CD4 cells was detected during cell expansion. CD4/CD8 ratio increased from 0.64±0.61 after CD4-CD8 cell selection, to 1.61±1.04 in the final product. A deeper analysis of this data revealed that in patients starting with a CD4/CD8 ratio <1, the proportion of CD4+ cells tended to increase during cell expansion, while in patients where a CD4/CD8 ratio >1 was obtained after cell selection, the proportion of CD4+ cells tended to decrease (FIG. 6B). Therefore, the difference in CD4/CD8 ratio (ΔCD4/CD8) before and after cell expansion was significantly different depending on the initial ratio. The efficiency of transduction differed between CD4+ and CD8+ subsets, as CD4 showed a significantly higher percentage of CAR+ cells (FIG. 6C).

In terms of T_{N}, T_{SCM}, T_{CM}, T_{E} and T_{EM} subsets, we observed a high degree of variability among patients' final products (FIG. 6D). This high variability is exemplified by the different level of CD45RA and CCR7 expression in samples from different patients (FIG. 6E) and cannot be attributed to the different diseases. Within the CAR+ T-cells of the final product, memory phenotypes (CM and EM) predominated in the vast majority of patients. Average percentage and SD for each subpopulation in the CAR+ cells of the final product is as follows: T_{N}: 7.71±13.9, T_{SCM}: 5.26±12.0, T_{CM}: 31.01±16.7, T_{EM}: 35.11±17.7 and T_{E}: 4.2±9.5. Analysis of CD4 and CD8 cells separately showed that CD8 cells have a more T_{N}, T_{SCM} and T_{CM} phenotype than CD4 cells. We also analyzed how these subsets varied during ex vivo cell expansion by comparing T-cell subsets in the initial (after CD4-CD8 cell selection) and in the final product, and if CAR expression influenced T-cell subpopulations (CAR− v.s. CAR+ cells). As shown in FIG. 6D, we observed a robust increase in the proportion of T_{CM} during cell expansion while T_{N} and T_{EFF} cells decreased. These changes in T-cell subsets can be attributed to a decrease in CD45RA expression which is expected upon cell activation (FIG. 6E).

No statistically significant changes in the T-cell subsets were detected between CAR− and CAR+ cells in the final product, although a further increase in T_{CM}, and consequent decrease in T_{EFF} cells, was observed in CAR+ cells compared to CAR− (FIG. 6D). Consistently, a small increase in CCR7 expression was also detected in CAR+ cells v.s. CAR− cells (FIG. 6E). The impact of CAR expression on CCR7 was further explored in independent small-scale expansions (see next section). The changes in expression of CD27, CD28 and CD95 were also assessed by flow cytometry. CD95 was increased during cell expansion and CD27 decreased. CD28 did not show significant changes during expansion, although presented a higher expression in CAR+ compared to CAR− cells.

### Example 2.2.5. Small-Scale CAR T Cell Expansions

To further evaluate the impact of culture conditions or CAR expression on the proportion of CD4/CD8 ratio or T-cell phenotype, cell expansions from patients' selected cells were repeated in a small scale experiment, under different conditions. We selected 6 of the patients (3 adult ALL and 3 NHL) from which frozen leftover cells after CD4-CD8 cell selection were available. We expanded patients' cells in 4 different conditions: (1a) IL2-Untransduced T-cells, (1b) IL2-CAR T-cells, (2a) IL7/IL15-Untransduced T-cells, (2b) IL7/IL15-CAR T-cells. Cells expanded between 17 and 100 times over a period of 11 days. CAR19 transduced T-cells expanded less (or slowly) compared to untransduced counterparts, and IL2 grown cells expanded more than IL7/IL15 (in both untransduced and CAR19 conditions). Cell transduction or cytokines used did not condition CD4/CD8 ratio in a consistent way. However, as detected previously in the products expanded using the Prodigy system, in patients starting with CD4/CD8 ratio>1 (T04 and T34), the ratio tended to decrease, while in patients starting with CD4/CD8 ratio<1 (T02, T15, T22 and T34), the ratio tended to increase. Indeed, since the expansions were maintained for longer in the small-scale expansions than in the Prodigy system, we observed that the ratio CD4/CD8 may fluctuate in a more or less pronounced way, but it tends to CD4/CD8=1 if the cells are cultured for longer periods of time.

Interestingly, significant differences were found in terms of T-cell subsets depending on the culture conditions. The cytokines used in the growth media did not provide significant differences in terms of the different subsets in this series of patients. However, a significant and consistent difference was appreciated in CAR19 expressing cells v.s. untransduced T-cells for almost all subsets. CAR19 transduction resulted in a much higher percentage of T_{N}, T_{SCM} and T_{CM} subsets independently of the cytokine used in the culture media. By contrary, T_{EM} cells were decreased in the CAR19+ cells compared to the untransduced samples. In this case, no difference in CD45RA MFI between untransduced and CAR19+ cells was observed that can account for the decrease in T_{N} and T_{SCM}, since in both conditions, cells were activated and proliferated ex vivo. However, we observed a significant increase in CCR7 expression in CAR19+ cells compared to untransduced cells. This increase explains a higher percentage of T_{N}, T_{SCM} and T_{CM} subsets and lower T_{EM}. Increase in CCR7 expression upon 4-1BB activation has been previously described in monocytes and also proposed for CAR T-cells. To test if 4-1BB activation is responsible for the increase in CCR7 we observe in the CAR+ cells, we modified our CAR construct by changing the co-stimulatory domain to CD28. T-cells from a healthy donor were then left untransduced or transduced with the 4-1BB- or CD28-containing CARs and expanded in vitro for 10 days. Again, we observed an increase in CCR7 expression in the CAR-positive fraction of the cells transduced with the 4-1BB-containing construct, compared to untransduced cells or CD28-containing CAR+ cells. As expected, percentage of T_{CM} cells is also higher in 4-1BB-containing CAR+ cells.

Finally, the functionality of CAR T-cells manufactured with the Prodigy system and small-scale expansions was also compared. For this comparison, cells from 3 patients expanded with IL-7/IL-15 were used. The production of pro-inflammatory cytokines, cytotoxic potential and T-cell expansion was measured after adjusting for the same percentage of CAR+ cells. Production of IFNγ and TNFα was measured after co-culture of CAR T-cells with NALM6 at 1:1 ratio, at 4 h time-point. Level of these two cytokines was measured both by intracellular staining and cytokines present in the media, yielding consistent results. Cells manufactured in the Prodigy system consistently produced slightly more IFNγ and TNFα than cells manufactured in small-scale expansions. However, these differences were not statistically significant. In terms of cytotoxic potential, cells produced with both methods showed comparable results. Finally, T-cell expansion upon repeated challenges with fresh target cells (NALM6) was slightly higher in cells manufactured with the Prodigy system than with small-scale expansions, although it did not reach statistical significance. Therefore, we conclude that cells manufactured with the Prodigy system are functionally comparable, or even slightly more active, than those produced in small-scale expansions.

Taking all this data together, we conclude that ex vivo cell expansion causes a loss of T_{N} and T_{EFF}, which is observed in both the Prodigy system and small-scale expansions. On the contrary, T_{CM} cells are largely accumulated, both by ex vivo expansion and as a result of CAR expression (in CARs containing 4-1BB as a co-stimulatory domain). Cells produced in Prodigy system are functionally similar to those produced in small-scale expansions.

### Example 3. Cell Therapy in Patients with CD19+ Relapsed/Refractory Malignancies

### Example 3.1. Material and Methods

### Example 3.1.1. Patient Population

The study carried out was a single-arm, multicenter, open-label pilot study evaluating the safety and efficacy of CAR cells of the invention in patients with R/R B-cell malignancies. Eligible patients had to have all of the following: (i) CD19-positive B-cell malignancy, including ALL, DLBCL, chronic lymphocytic leukemia (CLL), follicular lymphoma or mantle-cell lymphoma; (ii) age from 2 to 80 years; (iii) ECOG performance status 0-2; (iv) estimated life expectancy from 3 months to 2 years; and (v) adequate venous access. Key exclusion criteria included history of malignancy unless it had been in remission for more than 3 years; severe renal, hepatic, pulmonary or cardiac impairment; active immunosuppressive therapy; HIV infection; active HBV or HCV infection; and active infection requiring systemic therapy. Of note, neither CNS involvement nor prior alloHCT were exclusion criteria for this trial. All patients provided written, informed consent. The Spanish Medicines Agency (AEMPS) and Institutional Review Boards/Ethics Committees of each study site approved the trial, which was conducted in accordance with the principles of the Declaration of Helsinki.

### Example 3.1.2. Study Design, Procedures and Treatment

The primary endpoint was safety as determined by procedure-related mortality and grade 3-4 toxicity at day +100 and one year. Adverse events (AEs) of special interest were cytokine release syndrome (CRS), neurotoxicity (currently known as immune effector-cell associated neurotoxicity syndrome [ICANS]) and second neoplasia. AEs were graded according to common terminology criteria (CTC), version 4.0. For CRS, we used a grading system. Secondary endpoints included objective response rate as per NCCN, Lugano or IWCLL criteria; progression-free survival (PFS), overall survival (OS), duration of response (DOR), B-cell aplasia duration, and impact of therapy on quality of life.

Before infusion of CAR cells of the invention, patients received fludarabine at 30 mg/m²/day plus cyclophosphamide at 300 mg/m²/day on days −6, −5, and −4. On day 0, patients received a single intravenous infusion of CAR cells of the invention at a dose of 0.5-5 ×10⁶ cells/kg (later amended to fractionated administration, see below).

The original sample size was 10 patients (cohort 1). Five months after study initiation, a major amendment increased the sample size to 39 patients and allowed patients with either normal B-cell recovery within 3 months (early B-cell recovery), CD19-positive disease relapse or CD19-positive refractory disease to receive a second dose of CAR cells of the invention (cohort 2). Twelve months after study initiation, with 19 patients already recruited, a second major amendment increased the sample size to a total of 54 patients (cohort 3) and mandated the fractionated administration of CAR cells of the invention (10%, 30% and 60% of the total dose) contingent on the lack of CRS after the first and/or second fraction, and also the early administration of tocilizumab in patients with grade 2 CRS. This second amendment was motivated by 3 cases of grade 5 toxicity.

### Example 3.1.3. Statistical Analysis

The statistical analysis was purely descriptive, with adverse events and response rates presented with 95% exact Clopper-Pearson confidence intervals. Procedure-related mortality (PRM) was calculated as a cumulative incidence considering disease relapse as a competing event. OS, PFS, DOR and persistence of B-cell aplasia, were plotted using the Kaplan-Meier method. The impact of persistence of B-cell aplasia on PFS was evaluated using the Mantel-Byar method. All statistical analyses were performed using SAS version 9.4 (SAS Institute, Cary, NC) and R version 3.6 (R Foundation for Statistical Computing, Vienna, Austria).

### Example 3.2. Results

### Example 3.2.1. Baseline Characteristics

Fifty-eight patients were enrolled in the study, but four of them never made it through the screening phase: two patients did not comply with inclusion/exclusion criteria and two were eventually referred for therapy with commercial products that became available at the time. Out of the remaining 54 patients, 47 received therapy with CAR cells of the invention, 19 in cohorts 1-2 (single dose infusion) and 28 in cohort 3 (fractionated infusion). These 47 patients (modified full analysis set [mFAS]) were diagnosed with ALL (38), DLBCL (4, one of them a Richter's transformation from CLL), primary mediastinal B-cell lymphoma (2), follicular lymphoma (2) and CLL (1). Baseline characteristics of patients included in the mFAS are shown in Table 6.

TABLE 6

| | | Cohort 3 | |
|---|---|---|---|
| | Cohorts 1 and 2 | (Fractionated | All |
| Characteristic | (Single Infusion) | Infusion) | Patients |
| Acute lymphoblastic | n = 15 | n = 23 | n = 38 |
| leukemia | | | |
| Age (y), median (range) | 20 (3-35) | 29 (4-67) | 24.5 (3-67) |
| Female sex, n (%) | 4 (27) | 10 (44) | 14 (37) |
| ECOG performance | 4 (31) | 5 (22) | 9 (25) |
| status ≥1, n (%) | | | |
| Prior regimens, median | 4 (3-10) | 4 (2-8) | 4 (2-10) |
| (range) | | | |
| Prior inotuzumab, n (%) | 4 (27) | 9 (39) | 13 (34) |
| Prior blinatumomab, | 3 (20) | 6 (26) | 9 (24) |
| n (%) | | | |
| Prior allogeneic HCT, | 11 (73) | 22 (96) | 33 (87) |
| n (%) | | | |
| Bone marrow blast cell | 10 (67) | 7 (30) | 17 (45) |
| count ≥5%, n (%) | | | |
| Non Hodgkin's | n = 3 | n = 5 | n = 8 |
| lymphoma | | | |
| Histology: | | | |
| Diffuse large B cell | 1 (33.3) | 3 (60) | 4 (50) |
| lymphoma | | | |
| Primary mediastinal B | 2 (66.6) | 0 | 2 (25) |
| cell lymphoma | | | |
| Follicular lymphoma | 0 (0) | 2 (40) | 2 (25) |
| Age (y), median (range) | 25 (19-51) | 50 (22-62) | 47.5 (19-62) |
| Female sex, n (%) | 1 (33) | 2 (40) | 3 (37.5) |
| ECOG performance | 1 (33) | 3 (60) | 4 (50) |
| status ≥1, n (%) | | | |
| Prior regimens, median | 6 (5-7) | 6 (4-9) | 6 (4-9) |
| (range) | | | |
| Prior allogeneic/ | 3 (100) | 1 (20) | 4 (50) |
| autologous HCT, n (%) | | | |

HCT, hematopoietic cell transplantation;
NA, not applicable/available.

Median age was 26 years (range, 3-67), and 17 patients (36%) were female. The data cutoff date was Nov. 5, 2019, when all infused patients had a minimum follow-up of 100 days or had experienced disease relapse or death. At that time, the median follow-up for survivors was 5.48 months (range, 1.87-23.6) from infusion of CAR cells of the invention.

Out of 54 patients who proceeded to apheresis, 47 (87%) and 7 (13%) required one and two procedures, respectively. All infused patients received fludarabine+cyclophosphamide lymphodepletion and received CAR cells of the invention a median of 54 days (range, 34-215) after study inclusion. The original target dose ranged from 0.5 to 5 ×10⁶ of CAR cells of the invention (cells/kg), with the condition imposed by the AEMPS that the first patient had to receive the minimum dose (0.5 ×10⁶ CAR cells of the invention; cells/kg). In cohort 3, one patient received 0.4 ×10⁶ CAR cells of the invention (cells/kg) (i.e. the last fraction was omitted) due to CRS.

### Example 3.2.2. Toxicity

All adverse events (AEs) occurring from study inclusion, even before infusion of CAR cells of the invention, were graded and reported. Grade ≥3 AEs were documented in 68.4% of patients with ALL and 75% of patients with NHL at day +100, whereas serious AEs (SAEs) were observed in 44.7% and 50% of patients with ALL and NHL, respectively (Table 7). Procedure-related mortality (PRM) at day +100 was 7.9% (95% confidence interval [CI] 1.7-21.4%) for patients with ALL and 0% for patients with NHL. Suspected unexpected serious adverse reactions (SUSARs) were observed in four patients: two patients who developed lethal CRS and one patient who died of pseudomembranous colitis while recovering from grade 4 CRS. These three patients, belonging to cohorts 1-2, motivated the second major amendment of the study as previously described. The fourth SUSAR was reported in a patient with follicular lymphoma from cohort 3 who developed grade 4 toxic epidermal necrolysis while recovering from grade 2 CRS.

Regarding AEs of special interest, CRS was reported in 55.3% (13.2% grade ≥3) and 87.5% (25% grade ≥3) of patients with ALL and NHL, respectively. In patients with ALL, we observed a marked reduction in the rate of grade ≥3 CRS after the second amendment, dropping from 26.7% (cohort 1-2) to 4.3% (cohort 3) (Table 7).

TABLE 7

| | | Cohort 3 | |
|---|---|---|---|
| | Cohorts 1 and 2 | (Fractionated | All |
| | (Single Infusion) | Infusion) | Patients |
| Acute lymphoblastic | n = 15 | n = 23 | n = 38 |
| leukemia | | | |
| Grade ≥ 3 adverse | 11 (73.3) | 15 (65.2) | 26 (68.4) |
| events, n (%) | | | |
| Severe adverse events, | 8 (53.3) | 9 (39.1) | 17 (44.7) |
| n (%) | | | |
| Grade ≥3 CRS, n (%) | 4 (26.7) | 1 (4.3) | 5 (13.2) |
| Grade ≥3 ICANS, | 1 (6.7) | 0 (0.0) | 1 (2.6) |
| n (%) | | | |
| Grade ≥3 s | 1 (6.7) | 0 (0.0) | 1 (2.6) |
| malignancies, n (%) | | | |
| Non-Hodgkin's | n = 3 | n = 5 | n = 8 |
| lymphoma | | | |
| Grade ≥3 adverse | 3 (100) | 3 (60) | 6 (75) |
| events, n (%) | | | |
| Severe adverse | 3 (100) | 1 (20) | 4 (50) |
| events, n (%) | | | |
| Grade ≥3 CRS, n (%) | 1 (33.3) | 1 (20) | 2 (25) |
| Grade ≥3 ICANS, | 0 (0) | 0 (0) | 0 (0) |
| n (%) | | | |
| Grade ≥3 s | 0 (0) | 0 (0) | 0 (0) |
| malignancies, n (%) | | | |

CRS, cytokine release syndrome;
ICANS, immune effector cell-associated neurotoxicity syndrome.

Moreover, grade ≥3 ICANS was only observed in 1 (2.6%) patient with ALL. The only grade ≥3 second malignancy observed in the study was myelodysplasia in a 7-year-old girl diagnosed with ALL who had already received 6 lines of therapy, including IO and alloHCT. This patient has recently undergone a second alloHCT for this cause.

Globally speaking, the most common AEs in patients with ALL where neutropenia (97.4%), anemia (84.2%), hypogammaglobulinemia (78.9%), thrombocytopenia (76.3%) and lymphopenia (73.7%). Liver toxicity was also frequent, including increased AST (50%), increased ALT (47.4%), increased GGT (39.5%) and increased alkaline phosphatase (36.8%), mostly in patients with prior alloHCT (. Similar figures were observed in patients with NHL. Two patients with ALL (2/38, 5%) with prior history of alloHCT and IO therapy developed severe hepatic sinusoidal obstruction syndrome (SOS) that resolved with conventional supportive care.

### Example 3.2.3. Efficacy

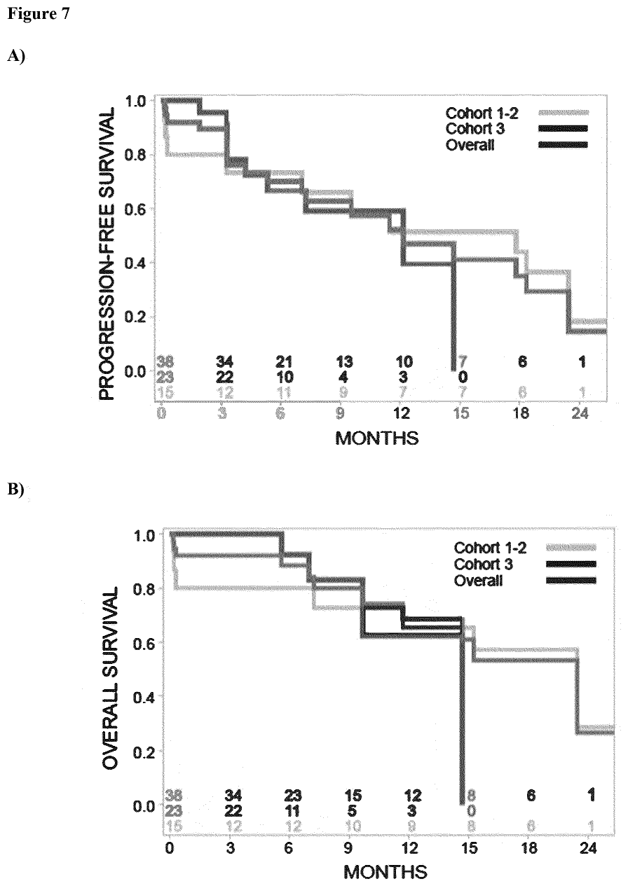

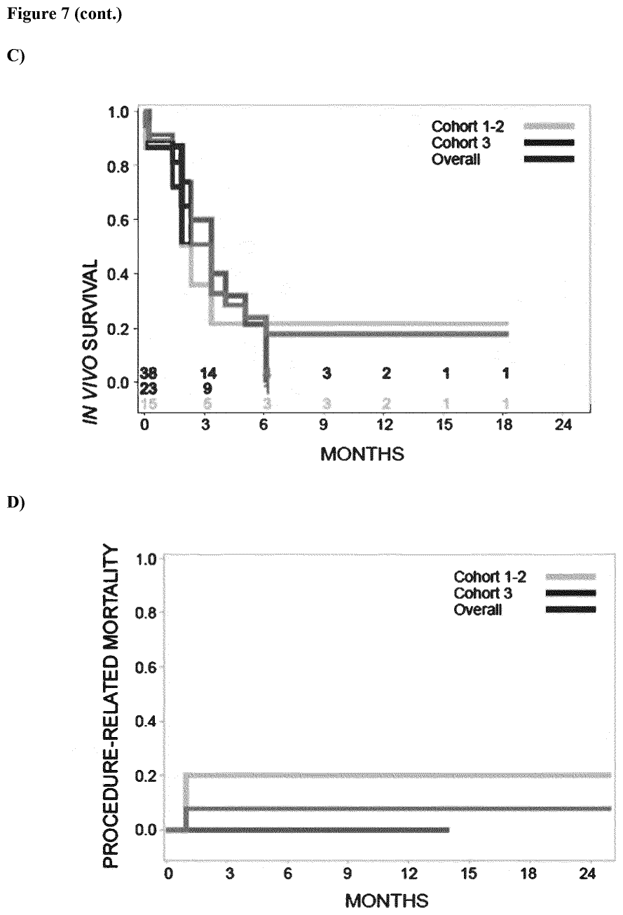

In patients with ALL, the measurable residual disease (MRD)-negative complete response rate (CRR) was 71.1% (95% CI 54%-85%) at day +100. All evaluable patients (i.e. excluding those who died prematurely) developed absolute B-cell aplasia that lasted for a median of 100 days (95% CI 56-100 days). PFS was 47% (27-67%) at one year for the whole ALL cohort, while OS was 68.6% (49-88%) at 1 year (78% for children, 65% for adults) (FIG. 7). The median DOR, considering only patients who responded to therapy by day +100, was 14.8 months. Out of 15 patients with progressive disease after infusion of CAR cells of the invention, tumor cells expressed CD19 in 13 (87%), while 2 (13%) were CD19-negative. There was no association between persistence of B-cell aplasia and PFS (P=0.33, Mantel-Byar test). Human anti-murine antibodies (HAMAs) were detected in 9/36 (25%) patients with ALL, three of them before the infusion of CAR cells of the invention. There was no association between the occurrence of HAMAs and loss of B-cell aplasia or disease relapse.

Subgroup analyses according to type of administration (cohort 1-2 vs. cohort 3) and age are depicted in Table 8.

DOR Median (95% CI) 1-year OS Median (95% CI) 1-year

| | | RD-CRR | MRD-GRR | PES Median | DOR Median | OS Median |
|---|---|---|---|---|---|---|
| | | at day +28, | at day +100; | (95% CI) 1-year | (95% CI) 1-year | (95% CI) 1-year |
| Population | n | CI | rate (95% CI) | rate (95% CI) | rate (95% CI) | rate (95% CI) |
| Total | 38 | 84.2 (69-94) | 71.1 (54-85) | 12.0 mo (4.2-20.2) 47% | 14.8 mo (6.0-NA) 59% | 20.2 mo (10.4-NA) 69% |
| | | | | (27-67%) | (34%-83%) | (49%-88%) |
| Cohorts 1 and 2 | 15 | 73.3 (45-92) | 66.7 (38-88) | 17.6 mo (0.3-NA) 51% | 15.7 (3.6-NA) 70% | 20.2 mo (0.3-NA) 65.5% |
| (single dose) | | | | (25%-77%) | (42%-98%) | (41%-90%) |
| Cohort 3 | 23 | 91.3 (72-99) | 73.9 (52-90) | 12.0 mo (4.2-14.5) 39.5% | 8.7 mo (2.0-NA) NA | 14.5 mo (6.8-14.5) 62% |
| (fractionated) | | | | (4%-75%) | (NA-NA) | (24%-100%) |
| Age groups | | | | | | |
| ≤18 years | 11 | 81.8 (48-98) | 54.5 (23-83) | 18.1 mo (14.5-NA) 82% | NA mo (14.8-NA) 100% | NR (7.1-NA) 78% |
| | | | | (59%-100%) | (100%-100%) | (50%-100%) |
| >18 years | 27 | 85.2 (66-96) | 77.8 (58-91) | 9.4 mo (3.3-20.2) 34% | 8.7 mo (3.9-16.6) 46% | 20.2 mo (12.8-NA) 64.5% |
| | | | | (12%-57%) | (18%-75%) | (40%-89%) |
| ≤25 years | 19 | 78.9 (54-94) | 63.2 (38-84) | 17.6 mo (4.2-20.2) 64% | 14.8 mo (6.0-NR) 79% | 20.2 mo (14.5-NA) 82% |
| | | | | (40%-89%) | (51%-100%) | (63%-100%) |
| >25 years | 19 | 89.4 (67-99) | 78.9 (54-94) | 7.2 mo (3.2-NA) 25% | 8.7 mo (2.0-NR) 34% | NR (6.9-NA) 50.5% |
| | | | | (0%-52%) | (0%-70%) | (18%-84%) |

MRD-CRR, complete response rate with negative measurable residual disease;
PFS, progression-free survival;
DOR, duration of response;
OS, overall survival;
mo, months;
NA, not available.

It is important to highlight that the apparent lower response rate observed in the pediatric population is due to the early administration of a second dose of CAR cells of the invention before day +100 in two patients. Both patients were in MRD-negative CR by then but received the second infusion shortly before this timepoint. If we count them both as responders, the CRR for pediatric patients would be 72% instead of 55%, and the CRR of the entire population would be 76% instead of 71%.

In patients with NHL, the overall response rate at day +100 was 75% (35-97%), while the CRR was 50% (16-84%).

### Example 3.2.4. Fractionated Administration

The cell composition of the invention was administered to the patients. The first 15 patients received a single intra-venous infusion of 0.5-1×10⁶ cells/kg (adults) or 5×10⁶ cells/kg (children) on day 0. The following 38 patients received 1×10⁶ cells/kg regardless of age: the first fraction (around 10%) on day 0, followed by the second (around 30%) and third (around 60%) fraction. The second fraction was administered 24-48 hours after the first, and the third 24-48 hours after the second, only if the patient had no signs or symptoms of CRS. The reason for this proposal was three cases of fatal toxicity (two patients, aged 11 and 19, who died of refractory CRS, and one patient, age 35, who died of pseudomembranous colitis as a complication of grade 4 CRS.

Adverse events and response rates are presented with 95% exact Clopper-Pearson CIs. The possible association between CRS (all grades and grade ≥3), tumor burden at screening (<5% vs ≥5% blasts in the bone marrow (BM)) and type of administration (single dose vs fractionated) was assessed using Fisher's exact test. We also analyzed the impact on progression-free survival (PFS) and overall survival (OS) of the following variables: age (<25 vs >25 years), type of administration, tumor burden and loss of B-cell aplasia (BCA), the latter as a time-dependent covariate. PFS/OS curves were plotted using the Kaplan-Meier method for time-fixed covariates and the Simon-Makuch method for BCA loss. Landmark analyses were performed to identify the most appropriate timepoint for BCA loss. Univariate Cox regression was used to evaluate the impact of these covariates on PFS/OS, and those with BenjaminiHochberg adjusted p values lower than 0.1 were introduced into a multivariate Cox regression. Schönfeld residuals were used to check the proportional hazards assumption. Fifty-three patients with R/R ALL received therapy with the cell composition of the invention. The median age was 30 years (range, 3-68), while 24 (45%) patients were female. Patients received the cell composition of the invention a median of 55.5 days (range, 27-216) after inclusion, and the median vein-to-vein time (from apheresis to infusion) was 43 days (range, 21-190). The original target dose was infused to all except 3 (5.7%) patients who received 0.1-0.4×10⁶ cells/kg due to CRS. CRS was reported in 56.6% (95% CI 42.3%-70.2%) of patients, being grade ≥3 in 11.3% (95% CI 4.3% to 23%) and requiring treatment with tocilizumab and steroids in 20.7% and 11.3% of patients, respectively. Patients with ≥5% lymphoblasts in the BM had a higher incidence of CRS (any grade: 82% vs 39%, p=0.0022; grade ≥3: 27% vs 0%, p=0.0036) compared with those with <5% lymphoblasts. Moreover, the incidence and severity of CRS was also associated with single dose versus fractionated administration of the cells (any grade: 87% vs 45%, p=0.0064; grade ≥3: 27% vs 5%, p=0.047). Neurotoxicity was observed in 13.2% (95% CI 5.5% to 25.3%) of patients, with one self-limited grade ≥3 occurrence (1.9%). No new second malignancies have been reported apart from a previously notified case of myelodysplasia (1/53; 1.9%).

The safety profile was comparable to that of similar products, with grade ≥3 CRS/neurotoxicity rates lower than 5%. 2 6 Moreover, both fractionated administration and tumor burden were significantly associated with the incidence of CRS, in keeping with similar studies. Of note, two patients enrolled in the CUP experienced grade ≥3 CRS with the first fraction (0.1×10⁶ cells/kg), but successfully recovered after treatment with tocilizumab. Both patients had a high tumor burden (>90% blasts in the BM) at study inclusion, and yet they could receive therapy while avoiding irreversible toxicity.

The measurable residual disease (MRD)-negative CR rate was 88.6% (95% CI 77.0% to 95.7%) at day +28% and 79.2% (95% CI 65.9% to 89.2%) at day +100. All three patients who received less than 1×10⁶ cells/kg due to toxicity achieved an MRD-negative CR. All evaluable patients (n=50) developed absolute BCA that lasted for a median of 4.2 months (95% CI 3.32 to 7.53 months). PFS was 50.9% (95% CI 38.4% to 67.4%) and 32.9% (95% CI 20.6% to 52.6%) at one and 2 years, respectively, while the 1-year and 2-year OS were 70.2% (95% CI 58.1% to 84.8%) and 53.9% (95% CI 40.5% to 71.8%). Progressive disease has occurred in 27 (50.9%; 95% CI 36.8 to 64.9%) patients at a median of 5.3 (range, 0.2-23.1) months. Tumor cells expressed CD19 in 24 (89%) of these relapses, while three (11%) were CD19-negative. The cells served as a bridge to alloHCT in three (6%) patients. Second infusions were documented in nine patients (three more than previously reported: four due to CD19+ relapse and five in patients with early BCA loss). These resulted in transient responses and brief periods of BCA, but one of these responses allowed the patient to receive a second alloHCT.

By univariate analysis, only two variables had a potential impact on PFS: tumor burden (<5% vs ≥5% lymphoblasts in the BM at screening), with a 2-year PFS of 52.5% (95% CI 36.4% to 75.7%) vs 10.7% (95% CI 2.1% to 54.4%) and an HR of 2.14 (95% CI 1.04 to 4.42) for patients with 5% or more blasts (adjusted p=0.077). On the other hand, loss of BCA had an HR of 4.41 (95% CI 1.59 to 12.2), adjusted p=0.0172. Both variables (tumor burden and loss of BCA) were also confirmed in the multivariate model, with an HR of 2.05 (95% CI 1.004 to 4.17) for patients with 5% or more blasts at screening (p=0.0484) and an HR of 4.32 (95% CI 1.57 to 11.86) for patients with loss of BCA (p=0.0045). Regarding OS, none of the covariates evaluated had sufficient impact to justify a multivariate analysis. Seeing that BCA loss had such an impact on PFS, we performed a series of landmark analyses to identify the most appropriate cut-off for clinical practice. We chose 3 and 6 months as potential landmark times because the median time to BCA loss was 4.2 months in our series. According to these analyses, the 3-month time point was the closest to statistical significance (HR 1.83; 95% CI 0.82 to 4.11; p=0.15.

In conclusion, both tumor burden and BCA loss appeared to have a significant impact on PFS and could guide clinicians in the management of patients after cell infusion. The validity of the fractionated cell administration was also confirmed.

1. A method for treating CD19+ malignancies, comprising administering to a subject in need thereof a therapeutically effective amount of a composition comprising a plurality of cells, wherein the cells comprise a chimeric antigen receptor (CAR) which in turn comprises an antibody, F(ab′)2, Fab, scFab or scFv with a light chain variable region (VL) and a heavy chain variable region (VH), wherein said VH comprises HCDR1, HCDR2 and HCDR3 polypeptides and VL comprises LCDR1, LCDR2 and LCDR3 polypeptides, and wherein HCDR1 consists of the sequence SEQ ID NO: 1, HCDR2 consists of the sequence SEQ ID NO: 2, HCDR3 consists of the sequence SEQ ID NO: 3, LCDR1 consists of the sequence SEQ ID NO: 4, LCDR2 consists of the sequence SEQ ID NO: 5 and LCDR3 consists of the sequence SEQ ID NO: 6.

2. The method of claim 1, wherein a progressive fractionated administration of the cells is carried out wherein the percentage of cells administered is progressively increased in each consecutive fraction administered.

3. The method of claim 1, wherein a fractionated administration of the cells is carried out by administering a first fraction comprising around 10% of the total dose on day 0, followed by a second fraction comprising around 30% of the total dose and by a third fraction comprising around 60% of the total dose.

4. The method of claim 1, wherein the antibody, F(ab)2, Fab, scFab or scFv comprises a VL domain and a VH domain, wherein the VL domain consists of SEQ ID NO: 7 and the VH domain consists of SEQ ID NO: 8.

5. The method of claim 1, wherein the CAR, further comprises a transmembrane domain, a costimulatory signalling domain and/or an intracellular signalling domain.

6. The method of claim 1, wherein the hinge and transmembrane domain of the CAR consists of CD8a of SEQ ID NO: 9, the costimulatory signalling domain consists of 4-1BB of SEQ ID NO: 10, and the intracellular signalling domain consists of CD38 of SEQ ID NO: 11.

7. The method of claim 1, wherein the CAR comprises the SEQ ID NO: 12.

8. The method of claim 1, wherein the cells are T-cells or NK-cells.

9. The method of claim 1, wherein the composition is a pharmaceutical composition comprising pharmaceutically acceptable carriers or diluents.

10. The method of claim 1, wherein the CD19+ malignancy is one of the following: acute lymphoblastic leukaemia, non-Hodgkin's lymphoma or chronic lymphocytic leukaemia.

11. The method of claim 1, wherein a total dose is 0.5-5×10⁶ cells/Kg.

12. The method of claim 11, wherein the total dose is 1×10⁶ cells/Kg.


SEQUENCE LISTING (13 sequences)

SEQ ID NO: 1 - protein, 11 aa, Artificial sequence
  synthetic sequence
FAFSSYWMNWV

SEQ ID NO: 2 - protein, 10 aa, Artificial sequence
  synthetic sequence
GQIYPGDGDT

SEQ ID NO: 3 - protein, 9 aa, Artificial sequence
  synthetic sequence
RKRITAVIT

SEQ ID NO: 4 - protein, 15 aa, Artificial sequence
  synthetic sequence
RASESVDNFGNSFMH

SEQ ID NO: 5 - protein, 9 aa, Artificial sequence
  synthetic sequence
IYIASNLES

SEQ ID NO: 6 - protein, 10 aa, Artificial sequence
  synthetic sequence
HQNNEDPLTF

SEQ ID NO: 7 - protein, 113 aa, Artificial sequence
  synthetic sequence
TGNIVLTQSPASLAVSLGQRATISCRASESVDNFGNSFMHWYQQKSGQPPRLLIYIASNLESGVPARFSGSGSRTDFTLTIDPVEADDAATYYCHQNNEDPLTFGAGTKLELK

SEQ ID NO: 8 - protein, 124 aa, Artificial sequence
  synthetic sequence
HSQIQLQQSGAELVRPGSSVKISCKASGFAFSSYWMNWVKQRPGQGLEWIGQIYPGDGDTKYNVKFRGKATLTADESSSTAYIQLTSLTSEDSGVYFCARKRITAVITTVFDVWGAGTTVTVSS

SEQ ID NO: 9 - protein, 69 aa, Artificial sequence
  synthetic sequence
TTTPAPRPPTPAPTIASQPLSLRPEACRPAAGGAVHTRGLDFACDIYIWAPLAGTCGVLLLSLVITLYC

SEQ ID NO: 10 - protein, 42 aa, Artificial sequence
  synthetic sequence
KRGRKKLLYIFKQPFMRPVQTTQEEDGCSCRFPEEEEGGCEL

SEQ ID NO: 11 - protein, 113 aa, Artificial sequence
  synthetic sequence
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 12 - protein, 481 aa, Artificial sequence
  synthetic sequence
TGNIVLTQSPASLAVSLGQRATISCRASESVDNFGNSFMHWYQQKSGQPPRLLIYIASNLESGVPARFSGSGSRTDFTLTIDPVEADDAATYYCHQNNEDPLTFGAGTKLELKGGGGSGGGGSGGGGSGGGGSHSQIQLQQSGAELVRPGSSVKISCKASGFAFSSYWMNWVKQRPGQGLEWIGQIYPGDGDTKYNVKFRGKATLTADESSSTAYIQLTSLTSEDSGVYFCARKRITAVITTVFDVWGAGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 13 - DNA, 2560 bp, Artificial sequence
  synthetic sequence
cgcctttttccggagggtgggggagaaccgtatataagtgcagtagtccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcatgagcctcatcccgaatggctctgcctgtcactggactgctgctgtcactggggctgctgctgcacgccgcaagaccaaccggtaacatcgtgctgactcagagcccagcatccctggccgtctcactgggacagcgggctaccatcagctgcagagcaagcgagtccgtggacaacttcggaaattcctttatgcactggtaccagcagaagtctggccagccccctcgactgctgatctatattgcctctaacctggagagtggcgtgccagctaggttctctggcagtgggtcacgcacagactttaccctgacaattgatcccgtcgaagccgacgatgccgctacctactattgccaccagaacaacgaggacccactgactttcggagcagggacaaaactggaactgaagggtggaggcgggagcggaggaggcgggtcgggaggaggaggatcgggtggcggaggctcacattctcagattcagctgcagcagtcaggggcagagctggtgcggccagggtcctcagtcaagattagttgtaaggcttcaggctttgctttcagctcctactggatgaactgggtgaaacagcgaccaggacagggactggagtggatcggacagatctaccccggagacggcgatacaaagtataatgtcaaatttcggggcaaggccaccctgacagctgacgagtctagttcaaccgcctacatccagctgacttcactgaccagcgaagattccggcgtgtatttctgcgctcgaaagcggatcactgcagtcattaccacagtgtttgacgtgtggggggcaggaactaccgtgaccgtcagctccacaactacgccggcgccgagaccacctacacctgcaccaactattgcctctcagccactgagtctgcgccccgaggcatgtcgacctgccgctggcggggctgtgcacaccaggggcctagacttcgcctgcgatatctatatttgggctccactggcaggaacctgtggcgtgctgctgctgtctctggtcatcacactgtactgcaaaagaggcaggaagaaactgctgtatattttcaagcagccctttatgagacctgtgcagacaactcaggaggaagacgggtgcagctgtaggttccctgaggaagaggaaggaggctgtgagctgcgcgtgaaattttctcggagtgcagatgccccagcttaccagcagggccagaaccagctgtataacgagctgaatctggggcggagagaggaatacgacgtgctggataagaggcgcgggcgagatccagaaatgggaggaaaaccccagcgacggaagaaccctcaggagggactgtacaatgaactgcagaaggacaaaatggcagaggcctattccgaaatcgggatgaaaggagaaagaaggcgcggcaaggggcatgatggcctgtatcagggactgtcaaccgcaacaaaagatacttatgatgctctgcacatgcaggctctgcccccgcggtaa